(19) United States
(12) Patent Application Publication
(10) Pub. No.: US 2025/0040684 A1
(43) Pub. Date: Feb. 6, 2025

(54) COSMETIC PACKAGING ARTICLE COMPRISING A SOLID COMPOSITION

(71) Applicant: L'Oreal, Paris (FR)

(72) Inventors: Damien Drillon, Saint Ouen (FR); Christelle Morvan, Saint Ouen (FR); Andreia Teixeira, Saint Ouen (FR)

(73) Assignee: L'Oreal, Paris (FR)

(21) Appl. No.: 18/716,715

(22) PCT Filed: Dec. 21, 2022
(86) PCT No.: PCT/EP2022/087357

§ 371 Date: Jun. 5, 2024

(30) Foreign Application Priority Data
Dec. 30, 2021 (FR) FR2114692

(51) Int. Cl. A45D 40/00; A45D 19/00; A61K 8/02; A61K 8/44; A61K 8/46; A61Q 5/02

(52) U.S. Cl. CPC A45D 40/00; A45D 19/005; A61K 8/02; A61K 8/44; A61K 8/466; A61Q 5/02; A61K 2800/87

(57) ABSTRACT

The present invention relates to a packaging article comprising a polymer-based multilayer envelope defining a cavity, and a solid composition based on one or more surfactant(s), said composition being contained in the cavity defined by the envelope. The invention also relates to a process for cosmetic treatment of keratin materials, in particular human keratin fibres such as the hair, using such a packaging article, and also to the use of said packaging article for washing and/or conditioning keratin materials, in particular human keratin fibres such as the hair.

The present invention relates to a packaging article comprising a polymer-based multilayer envelope defining a cavity, and a solid composition based on surfactant(s), said composition being contained in the cavity defined by the envelope. The invention also relates to a process for cosmetic treatment of keratin materials, in particular human keratin fibres such as the hair, using such a packaging article, and also to the use of said packaging article for washing and/or conditioning keratin materials, in particular human keratin fibres such as the hair.

In the field of hair hygiene, conventional products, such as shampoos or conditioners, are usually in more or less thickened liquid form. However, on account of their liquid texture, these products may have various drawbacks, and may notably prove to be difficult to measure out.

The reason for this is that the more liquid they are, the greater their tendency to escape between the fingers, making them difficult to measure out and leading to waste. These products may also escape from their packaging, which is a source of inconvenience to the consumer when these products come into contact with clothing or objects, for example when travelling.

In order to modify the texture of these products, and notably to make it more compact, thickeners are generally used. However, the addition of these compounds usually comes at the expense of the cosmetic effects of the compositions. Moreover, the use of these thicker compositions requires a lot of rinsing water in order to remove the surplus product on the fibres. Now, in many countries where access to water is restricted, the rinsing time and consequently the amount of water required to properly rinse off the product are key indicators of the working qualities of a composition.

In order to overcome some of these problems, novel solid cosmetic formulations, notably shampoos or conditioners, in the form of solid granules or powder, have been developed. However, these novel formulations are not always entirely satisfactory. Those which are in loose powder form may, indeed, pose problems of volatility, uptake and/or measuring out. Those which are in the form of agglomerates, for instance granules, may have a tendency to disintegrate or break down with difficulty in the presence of water and, in the case of shampoos, do not always make it possible to obtain a rapid start of foaming and/or a satisfactory abundance of foam, having a negative impact on their use and their spreading on the keratin fibres. They may also be difficult to remove on rinsing and may occasionally even leave residues on the fibres, which the consumer finds unpleasant.

Shampoos and conditioners in powder or particle form may lose fluidity during storage due to the agglutination of the individual solid particles with one another, which may have a negative impact on the working qualities.

These formulations may also not be entirely satisfactory in terms of cosmetic performance qualities, notably in terms of suppleness, feel, softness, sheen and disentangling.

Thus, there is a real need to provide a packaging article comprising a composition in solid form and having an improved environmental profile, notably linked to the reduction in the use of plastic and to the reduction of transport-related pollution.

The packaging article must also be easy to grasp, must allow easy storage of the composition that it contains, and must allow easy measuring out of the composition.

The packaging article and the composition that it contains must dissolve and break down easily. The composition must also, in the case notably of shampoos, have good foaming properties, notably in terms of the start of foaming and the foam abundance and density, but it must also rinse out quickly without leaving residues on the keratin fibres.

In the case of shampoos, the composition must also have good detergent power.

The composition must also confer satisfactory cosmetic properties, notably in terms of suppleness, feel, softness, sheen and disentangling, in particular when it is in the form of a hair care composition, such as a conditioner, but also a shampoo-conditioner.

It has now been discovered that a particular packaging article comprising a solid composition comprising less than 10% by weight of water, relative to the total weight of the composition, and one or more surfactants, notably a particular combination of surfactants, makes it possible to achieve the objectives set out above, and notably to be easy to grasp and to dissolve and break down easily.

A subject of the present invention is thus a packaging article comprising:
an envelope defining at least one cavity, the envelope being composed of at least two layers assembled together in order to form just one unit film,
the first layer comprising at least 60% by weight, relative to the total weight of the first layer, of one or more compounds chosen from cellulose-based polymers, plant fibres and mixtures thereof, and
the second layer comprising at least 75% by weight, relative to the total weight of the second layer, of one or more vinyl alcohol polymers,
a solid composition comprising less than 10% by weight of water relative to the total weight of the composition, and one or more surfactants,
it being understood that said solid composition is in at least one of the cavities defined by the envelope.

This packaging article notably makes it possible to solve the problems of measuring out of the solid composition. It also facilitates its storage and transportation. In particular, the packaging article of the invention affords better protection of the composition against moisture.

The packaging article of the invention dissolves easily and rapidly in water, and thus requires little water while being used.

Furthermore, this packaging mode is particularly advantageous, for example, when travelling or performing a sporting activity (lightened bags, limited risks of leakage, reduced waste).

The packaging article, when it comprises several cavities, can also make it possible to provide, for example, a shampoo and a conditioner in the same article.

In addition, this composition contained in the article breaks down rapidly on contact with water and makes it possible, where appropriate, to readily and quickly obtain a firm, creamy and abundant foam, the quality of which is comparable to that of the foam obtained with a conventional liquid shampoo composition. This foam can then be easily and uniformly spread on the keratin fibres.

The packaging article may also make it possible to obtain a final keratin fibre washing and/or conditioning composition that is more thickened in the hand, which may be in cream form. It may also act as a foam booster. Specifically, the volume of foam obtained after dilution of the packaging article may be greater than the volume of foam obtained after dissolution of the solid composition alone.

Moreover, the composition of the invention rinses out rapidly without leaving unpleasant residues on the fibres and gives them a natural, clean feel after rinsing. Fibres treated with the composition of the invention also have good cosmetic properties, notably in terms of softness, suppleness and feel. They also have good strand separation and are thus easier to disentangle.

The invention also relates to a cosmetic treatment process, notably for washing and/or conditioning keratin fibres, in particular human keratin fibres such as the hair, comprising a step of using at least one packaging article as defined above.

Other subjects, characteristics, aspects and advantages of the invention will become even more clearly apparent on reading the description and the example which follows.

In that which will follow, and unless otherwise indicated, the limits of a range of values are included in this range, in particular in the expressions “of between” and “ranging from . . . to . . . ”.

Moreover, the expression “at least one” used in the present description is equivalent to the expression “one or more”.

In addition, terms such as “first” or “second” used in the present application do not imply a sequence or an order, unless clearly indicated by the context.

### Packaging Article

The present invention notably relates to a packaging article comprising:
an envelope defining at least one cavity, the envelope being composed of at least two layers assembled together in order to form just one unit film,
the first layer comprising at least 60% by weight, relative to the total weight of the first layer, of one or more compounds chosen from cellulose-based polymers, plant fibres and mixtures thereof, and
the second layer comprising at least 75% by weight, relative to the total weight of the second layer, of one or more vinyl alcohol polymers.

Preferably, the packaging article is a cosmetic packaging article. The term “cosmetic packaging article” means an article that is suitable for cosmetic use; in particular for use of the packaging article on keratin materials, notably the hair and/or on the scalp. In particular, the packaging article makes it possible to wash and/or condition the keratin fibres, in particular human keratin fibres such as the hair.

Preferably, the packaging article according to the invention is water-soluble at a temperature of less than or equal to 35° C.

Preferably, the envelope of the packaging article according to the invention is water-soluble at a temperature of less than or equal to 35° C. Preferably, the first layer and/or the second layer of the envelope are water-soluble at a temperature of less than or equal to 35° C.

The term “water-soluble” means soluble in water, in particular in a proportion of at least 10 grams per litre of water, preferably at least 20 g/1, better still at least 50 g/1, at a temperature of less than or equal to 35° C. Thus, when water preferably having a temperature of less than 35° C. is added to the packaging article, the envelope will dissolve and release the solid composition present in one of the cavities of the envelope.

The term “temperature of less than or equal to 35° C.” means a temperature not exceeding 35° C. but greater than or equal to 0° C., for example ranging from more than 1 to 35° C., preferably from 5 to 30° C., more preferentially from 10 to 30° C. and better still from 15 to 25° C. It is understood that all the temperatures are given at atmospheric pressure (1 atm).

The packaging article may comprise one or more cavities, at least one of which contains the solid composition as defined hereinbelow. Preferably, the packaging article comprises only one cavity in which the solid composition is contained.

Advantageously, the envelope represents from 0.5% to 20% by weight, preferably from 1% to 15% by weight, more preferentially from 2% to 10% by weight, better still from 4% to 10% by weight, and even better still from 4% to 8% by weight, relative to the total weight of the packaging article.

Advantageously, the solid composition as defined hereinbelow represents from 80% to 99.5% by weight, preferably from 85% to 99% by weight, more preferentially from 90% to 98% by weight, better still from 90% to 96% by weight, and even better still from 92% to 96% by weight, relative to the total weight of the packaging article.

The weight ratio between the total weight of the solid composition of the invention and the total weight of the envelope advantageously ranges from 80/20 to 99/1, preferably from 85/15 to 98/2, and more preferentially from 90/10 to 97/3.

The envelope of the packaging article preferably has a breakup time, measured at 10° C., of between 1 and 7 seconds, more preferentially of between 2 and 6 seconds.

The Compound(s) Chosen from Cellulose-Based Polymers and Plant Fibres

The first layer of the packaging article according to the invention comprises one or more compounds chosen from cellulose-based polymers, plant fibres and mixtures thereof, present in a content of greater than or equal to 60% by weight, relative to the total weight of the first layer.

By way of plant fibres, use may be made of wood pulp.

By way of cellulose-based polymers, mention may be made of celluloses and cellulose derivatives, which may be anionic, cationic, amphoteric or non-ionic.

Cellulose ethers, cellulose esters and cellulose ether esters are distinguished among these cellulose derivatives.

Among the cellulose esters, mention may be made of inorganic esters of cellulose (cellulose nitrates, sulfates or phosphates), organic esters of cellulose (cellulose monoacetates, triacetates, amidopropionates, acetatebutyrates, acetatepropionates or acetatetrimellitates), and mixed organic/inorganic esters of cellulose, such as cellulose acetatebutyrate sulfates and cellulose acetatepropionate sulfates.

Among the cellulose ester ethers, mention may be made of hydroxypropylmethylcellulose phthalates and ethylcellulose sulfates.

Among the non-ionic cellulose ethers, mention may be made of alkylcelluloses such as methylcelluloses and ethylcelluloses (for example Ethocel Standard 100 Premium from Dow Chemical); hydroxyalkylcelluloses such as hydroxymethylcelluloses, hydroxyethylcelluloses (for example Natrosol 250 HHR sold by Aqualon) and hydroxypropylcelluloses (for example Klucel EF from Aqualon); mixed hydroxyalkyl-alkylcelluloses such as hydroxypropylmethylcelluloses (for example Methocel E4M from Dow Chemical), hydroxyethylmethylcelluloses, hydroxyethylethylcelluloses (for example Bermocoll E 481 FQ from Akzo Nobel) and hydroxybutylmethylcelluloses.

Among the anionic cellulose ethers, mention may be made of carboxyalkylcelluloses and salts thereof. Examples that may be mentioned include carboxymethylcelluloses, carboxymethylmethylcelluloses (for example Blanose 7M from Aqualon) and carboxymethylhydroxyethylcelluloses, and also the sodium salts thereof.

Among the cationic cellulose ethers, mention may be made of crosslinked or non-crosslinked quaternized hydroxyethylcelluloses. The quaternizing agent may notably be diallyldimethylammonium chloride (for example Celquat L200 from National Starch). Another cationic cellulose ether that may be mentioned is hydroxypropyltrimethylammonium hydroxyethyl cellulose (for example Ucare Polymer JR 400 from Amerchol).

Mention may also be made of celluloses or derivatives thereof, modified with groups comprising at least one fatty chain, such as alkyl, arylalkyl or alkylaryl groups or mixtures thereof in which the alkyl groups are of C8-C22; non-ionic alkylhydroxyethylcelluloses such as the products Natrosol Plus Grade 330 CS and Polysurf 67 (C₁₆ alkyl) sold by Aqualon; quaternized alkylhydroxyethylcelluloses (cationic) such as the products Quatrisoft LM 200, Quatrisoft LM-X 529-18-A, Quatrisoft LM-X 529-18-B (C₁₂ alkyl) and Quatrisoft LM-X 529-8 (C₁₈ alkyl) sold by Amerchol, the products Crodacel QM, Crodacel QL (C₁₂ alkyl) and Crodacel QS (C₁₈ alkyl) sold by Croda, and the product Softcat SL 100 sold by Amerchol; non-ionic nonoxynylhydroxyethylcelluloses such as the product Amercell HM-1500 sold by Amerchol; non-ionic alkylcelluloses such as the product Bermocoll EHM 100 sold by Berol Nobel.

Preferably, the compound(s) chosen from cellulose-based polymers, plant fibres and mixtures thereof are chosen from wood pulp, carboxymethylcelluloses, and also the sodium salts thereof, and mixtures thereof.

Preferably, the compound(s) chosen from cellulose-based polymers, plant fibres and mixtures thereof are present in the first layer in a content of greater than or equal to 70% by weight, more preferentially greater than or equal to 75% by weight, even more preferentially greater than or equal to 80% by weight, better still greater than or equal to 90% by weight and even better still greater than or equal to 95% by weight, relative to the total weight of the first layer.

Advantageously, the first layer has a thickness of between 50 and 125 μm, preferably between 60 and 85 μm, more preferentially between 65 and 80 μm.

Advantageously, the first layer has a water uptake of between 1.5% and 4.5%, preferably between 2% and 4%, and more preferentially between 2.5% and 3.5%, relative to the initial weight of the layer.

The method for measuring the water uptake is described in document WO 2017/218449. The water capacity is measured with a DVS (Dynamic Vapour Sorption) instrument of SPS-DVS type (model SPSx-\hat{ }-High load with permeability kit) from ProUmid. DVS uses gravimetric analysis to determine moisture sorption/desorption and is entirely automated.

The measurements are carried out at 20° C. The relative humidity (RH) is fixed at 35% (35% RH) for 6 h and then gradually brought to 50% (50% RH) over the course of 5 minutes. The relative humidity is then left at 50% for 12 hours. The total measurement time is 18 hours.

The water uptake (or % Dm) represents the relative variation in the mass relative to the initial weight of the layer or of the film, that is to say that 10% reflects a 10% increase in the weight of the layer or of the film relative to the initial weight.

The water uptake (or % Dm gained on the cycle at 50% RH during the fixed time of 12 hours at 20° C.) is calculated by the difference of the value % Dm at 50% RH (final value measured at 50% RH) minus % Dm at 35% RH (final value before increasing to 50% RH).

Advantageously, the first layer has a breaking strength of between 40 and 80 N, preferably between 50 and 70 N.

### Vinyl Alcohol Polymer(s)

The second layer of the packaging article according to the invention comprises one or more vinyl alcohol polymers, present in a content of greater than or equal to 75% by weight, relative to the total weight of the second layer.

The vinyl alcohol polymers of formula CH₂═CHOH result from the total or partial hydrolysis of vinyl acetate polymers CH₂═CHOC(O)CH₃.

Thus, the second layer preferably comprises one or more copolymers of vinyl alcohol and vinyl acetate.

The degree of hydrolysis of the vinyl acetate polymers is preferably greater than or equal to 75%, more preferentially greater than or equal to 80%.

The weight-average molecular mass of said polymer, measured by light scattering, is preferably less than or equal to 100 000 g/mol, more preferentially less than or equal to 80 000 g/mol, and even more preferentially less than or equal to 60 000 g/mol.

The polymer(s) of the second layer have a polydispersity index of preferably less than or equal to 2, more preferentially less than or equal to 1.5.

Preferably, the vinyl alcohol polymer(s) are present in the second layer in a content of greater than or equal to 80% by weight, more preferentially greater than or equal to 85% by weight, even more preferentially greater than or equal to 90% by weight, better still greater than or equal to 95% by weight, relative to the total weight of the second layer.

Advantageously, the second layer has a thickness of between 8 and 25 μm, preferably between 10 and 20 μm, more preferentially between 12 and 17 μm.

Advantageously, the second layer has a water uptake of between 2% and 6%, preferably between 3% and 5%, and more preferentially between 3.5% and 4.5%, relative to the initial weight of the layer.

Advantageously, the second layer has a breaking strength of between 5 and 20 N, preferably between 10 and 15 N.

### Polyols

The first layer and/or the second layer of the packaging article according to the invention can optionally also comprise one or more plasticisers, notably chosen from polyols.

The plasticiser(s) chosen from polyols that are optionally present in the first layer and/or the second layer are preferably chosen from glycerol, polyethylene glycols and mixtures thereof, and more particularly the product called PEG-6 or PEG-8 in the CTFA (International Cosmetic Ingredient Dictionary, Seventh Edition) publication.

Other polyols that may be used in the first layer and/or the second layer are those described in the section relating to the solid composition hereinafter.

Preferably, the molecular weight (MW) of said polyol(s) optionally present in the first layer and/or the second layer is between 50 and 350, more preferentially between 60 and 200, and even better still between 70 and 100.

When they are present in the first layer, the total content of the plasticiser(s) preferably ranges from 0.1% to 15% by weight, more preferentially from 0.5% to 10% by weight, and even more preferentially from 1% to 5% by weight, relative to the total weight of the first layer.

When they are present in the first layer, the total content of the polyol(s) preferably ranges from 0.1% to 15% by weight, more preferentially from 0.5% to 10% by weight, and even more preferentially from 1% to 5% by weight, relative to the total weight of the first layer.

When they are present in the second layer, the total content of the plasticiser(s) preferably ranges from 0.1% to 15% by weight, more preferentially from 0.5% to 10% by weight, and even more preferentially from 1% to 5% by weight, relative to the total weight of the second layer.

When they are present in the second layer, the total content of the polyols(s) preferably ranges from 0.1% to 15% by weight, more preferentially from 0.5% to 10% by weight, and even more preferentially from 1% to 5% by weight, relative to the total weight of the second layer.

Preferably, the first layer and/or the second layer comprise one or more plasticisers, notably chosen from polyols.

### Fillers

The first layer and/or the second layer of the packaging article according to the invention can optionally also comprise one or more fillers.

The fillers are defined and more thoroughly described in the section relating to the solid composition hereinafter.

The filler(s) that may be used in the first and/or the second layer can be chosen from mineral fillers, non-polymeric organic fillers, polymeric organic fillers, and mixtures thereof. Among the fillers that may be used, mention may particularly be made of starches, alginates and celluloses.

When they are present in the first layer, the total content of the filler(s) preferably ranges from 0.1% to 15% by weight, more preferentially from 0.5% to 12% by weight, and even more preferentially from 1% to 10% by weight, relative to the total weight of the first layer.

When they are present in the second layer, the total content of the filler(s) preferably ranges from 0.1% to 15% by weight, more preferentially from 0.5% to 12% by weight, and even more preferentially from 1% to 10% by weight, relative to the total weight of the second layer.

Preferably, the first layer and/or the second layer comprise one or more fillers.

Preferentially, the first layer comprises one or more plasticisers, notably chosen from polyols, and one or more fillers.

Preferentially, the second layer comprises one or more plasticisers, notably chosen from polyols, and one or more fillers.

The first layer and/or the second layer can optionally also comprise additional compounds, such as stabilizers and/or colouring agents (soluble dyes, insoluble pigments).

Advantageously, the first layer has a thickness greater than that of the second layer. Preferably, the total thickness of the unit film formed by the first layer and the second layer is greater than or equal to 60 μm, more preferentially greater than or equal to 65 μm, even more preferentially greater than or equal to 70 μm.

Advantageously, the water uptake of the second layer is greater than the water uptake of the first layer. Preferably, the difference between the water uptake of the second layer and the water uptake of the first layer is greater than or equal to 1%.

Advantageously, the breaking strength of the first layer is greater than the breaking strength of the second layer. Preferably, the difference between the breaking strength of the first layer and the breaking strength of the second layer is greater than or equal to 10 MPa, more preferentially greater than or equal to 20 MPa.

Advantageously, the ratio between the weight of the first layer and the weight of the second layer ranges from 25/75 to 75/25, preferably from 30/70 to 70/30, more preferentially from 35/65 to 65/35, even more preferentially from 65/35 to 55/45.

The first layer and the second layer are assembled together in order to form just one unit film. This unit film makes it possible to define the envelope of the packaging article according to the invention.

The term “film” is intended to mean notably a continuous layer preferentially formed from one or more polymers as defined above, in particular water-soluble polymers.

It is understood that the invention is not limited to an envelope formed by a single first layer and a single second layer. In particular, the envelope of the packaging article can comprise several first layers and/or several second layers. In these embodiments, the combination of the first and second layers is assembled so as to form just one unit film.

The main industrial methods for the production of polymer films are extrusion of a molten polymer, casting of a solution of a polymer onto a polished metal surface (in certain cases, the polymer solution is introduced into a precipitation tank), casting of a dispersion of the polymer onto a polished surface, and calendering.

The films that may be used according to the present invention may be chosen from film-multilayer film, film-paper (laminating) and film-coating.

During application by spraying, brushing or various industrial processes, the surface coatings undergo what is known as the formation of a film, and notably of film-coating. In the majority of the film-forming processes, a liquid coating of relatively low viscosity is applied to a solid substrate and is hardened as a solid adherent film based on high molecular weight polymer having the properties desired by the user.

The assembly of the different layers together can be carried out by applying heat, for example using a heating lamp or a heating roll.

Preferably, the second layer is adhesive. In this way, the first and the second layer can adhere together to form the unit film.

Particularly preferably, the second layer is adhesive on one of the faces thereof. In this case, the first layer is assembled on this adhesive face of the second layer.

In a first embodiment of the invention, the first layer is formed and then covered with the second layer.

In another embodiment of the invention, the second layer is formed and then covered with the first layer.

In these embodiments of the invention, the layer covered can be subjected to a treatment to promote adhesion of the layer which covers it, such as a corona and/or plasma surface treatment, the use of a tie layer between the layers of the present films, the application of an adhesive, and/or texturing methods.

Preferably, the film(s) defined by the layers are sealed so as to form one or more cavities which will comprise the solid composition defined hereinafter and will prevent it from escaping.

In one particular embodiment, the first layer forms the exterior and the second layer forms the interior of the envelope of the packaging article.

In another particular embodiment, the first layer forms the interior and the second layer forms the exterior of the envelope of the packaging article.

The packaging article, and also the envelope, may have any shape that is suitable for the intended use, for example a square, rectangular, round or oval shape. Preferably, it has a rounded geometry, for example in the form of a sphere, a disc or an oval, or else a square or parallelepipedal geometry preferably with rounded corners. The envelope preferably has dimensions allowing it to be taken up between at least two fingers. Thus, it may, for example, have an ovoid shape about 2 to 10 cm long and about 0.5 to 4 cm wide, or a circular disc shape about 2 to 10 cm in diameter, or a square shape with a side length of about 2 to 15 cm, or a rectangular shape with a length of about 2 to 25 cm, it being understood that it may have any other shape and size that are suitable for the intended use.

Preferably, the envelope may be of round shape with an inside diameter ranging from 2 to 7 cm, more preferentially from 4 to 5 cm; to which may be added the dimension of the edges (sealed part) which may range from 1 to 5 mm, better still from 2 to 4 mm; and a height ranging from 2 to 7 mm, preferentially from 3 to 5 mm.

The envelope may also be of square or rectangular shape with a length preferably ranging from 2 to 7 cm, more preferentially from 3 to 5 cm, and a width preferably ranging from 2 to 5 cm, more preferentially 2.5 to 4 cm; to which may be added the dimension of the edges (sealed part) which may preferably range from 1 to 5 mm, and more preferentially from 2 to 4 mm.

The area delimiting the cavity or cavities has an extent advantageously less than 625 cm², preferably between 0.025 cm² and 400 cm², more preferentially between 1 and 200 cm², better still between 2 and 50 cm² and even better still between 4 and 25 cm², so as to have optimized compacting of the composition. It has been observed that when the area of the article is within the above ranges, the compacting of the solid composition made of powder is lower and the transformation of the powder into a fluid composition in the hands is easier, without any formation of agglomerates.

Preferably, the height of the envelope is greater than or equal to 2 mm, more preferentially ranging from 2 to 10 mm, and better still from 3 to 7 mm.

The first layer and/or the second layer can optionally comprise indications in printed form or in the form of thermal relief, or be the subject of a stamp. Preferably, when they are present, said indications are on the layer forming the outer part of the envelope, and more preferentially on the external surface of said layer forming the outer part of the envelope.

### Solid Composition

The packaging article according to the invention also comprises a solid composition comprising less than 10% by weight of water relative to the total weight of the composition, and one or more surfactants, said composition being in at least one of the cavities defined by the envelope.

Preferably, the water content of the solid composition is less than or equal to 8% by weight, more preferentially less than or equal to 7% by weight, even more preferentially less than or equal to 5% by weight, better still less than or equal to 5% by weight, even better still less than or equal to 2% by weight, or even less than or equal to 1% by weight, relative to the total weight of the composition.

The solid composition according to the invention may be in the form of a powder, a paste, particles (for example spherical particles such as small balls or granules), a compressed tablet, a stick or a bar. Preferably, the composition according to the invention is in the form of a powder or of particles.

The term “powder” means a composition in pulverulent form, which is preferably essentially free of dust (or fine particles). In other words, the particle size distribution of the particles is such that the weight content of particles which have a size of less than or equal to 50 μm (content of fines), preferably less than or equal to 45 μm (content of fines) is advantageously less than or equal to 5% by weight, preferably less than 3% by weight and more particularly less than 1% by weight, relative to the total weight of particles (particle size evaluated using a Retsch AS 200 Digit particle size analyser; oscillation height: 1.25 mm/screening time: 5 minutes).

The term “paste” means a composition having a viscosity of greater than 5 poises (0.5 Pa·s) and preferably greater than 10 poises (1 Pa·s), measured at 25° C. and at a shear rate of 1 s⁻⁴; this viscosity possibly being determined using a cone-plate rheometer.

The term “particles” means small fractionated objects formed from solid particles that are aggregated together, of variable shapes and sizes. They may be in regular or irregular form. They may in particular be in spherical form (such as granules, granulates or beads) or in square, rectangular or elongated form such as sticks. Spherical particles are most particularly preferred.

Advantageously, the size of the powders or particles is, in its largest dimension, between 45 μm and 5 mm, more particularly between 50 μm and 2 mm, better still between 50 μm and 1 mm and even better still between 60 and 600 μm.

When the solid composition according to the invention is not in powder or particle form, it advantageously has a penetration force at 25° C. and 1 atm of greater than or equal to 200 g, preferably greater than or equal to 300 g, more preferentially greater than or equal to 400 g, and better still greater than or equal to 500 g. The penetration force is determined by penetrometry. The texture analysis measurements are performed at 25° C. using a Stable Micro Systems TA.XT Plus texturometer. The penetrometry experiments are performed with a metal rod equipped with a screwed end piece, said end piece being a P/2N needle of 2 mm for the top part, connected to the measuring head. The piston penetrates into the sample at a constant speed of 1 mm/s, to a depth of 5 mm. The force exerted on the piston is recorded and the mean value of the force is calculated.

The solid composition according to the invention can be in the form of a compressed solid composition, in particular compressed using a manual or mechanical press. Preferably, the hardness of the compressed solid composition is between 10 and 300 N, more preferentially between 15 and 200 N, and even better still between 15 and 100 N.

The density of the solid composition according to the present invention is preferably between 0.1 and 1, more preferentially between 0.2 and 0.8, and even better still between 0.3 and 0.6.

A given amount (mass, m) of powder is placed in a measuring cylinder. The measuring cylinder is then automatically tapped 2500 times. The volume (v) thus obtained is read on the measuring cylinder and the density (d) is then determined according to the formula d=m/v.

The surfactant(s) included in the solid composition may be chosen from anionic surfactants, amphoteric or zwitterionic surfactants, cationic surfactants and non-ionic surfactants and/or mixtures thereof.

### Anionic Surfactants

The solid composition contained in the packaging article according to the invention can comprise one or more anionic surfactants.

The term “anionic surfactant” means a surfactant including, as ionic or ionizable groups, only anionic groups.

In the present description, a species is termed as being “anionic” when it bears at least one permanent negative charge or when it can be ionized to a negatively charged species, under the conditions of use of the composition of the invention (for example the medium or the pH) and not comprising any cationic charge.

The anionic surfactants may be chosen from sulfate, sulfonate and/or carboxylic (or carboxylate) surfactants. Needless to say, a mixture of these surfactants may be used.

It is understood in the present description that:
the carboxylate anionic surfactants comprise at least one carboxylic or carboxylate function (—COOH or —COO⁻) and may optionally also comprise one or more sulfate and/or sulfonate functions;
the sulfonate anionic surfactants comprise at least one sulfonate function (—SO₃H or —SO₃⁻) and may optionally also comprise one or more sulfate functions, but do not comprise any carboxylate functions; and
the sulfate anionic surfactants comprise at least one sulfate function but do not comprise a carboxylate or sulfonate function.

The carboxylic anionic surfactants that may be used thus include at least one carboxylic or carboxylate function (—COOH or —COO⁻).

They may be chosen from the following compounds: fatty acids, acylglycinates, acyllactylates, acylsarcosinates, acylglutamates; alkyl-D-galactosideuronic acids, alkyl ether carboxylic acids, alkyl(C₆-C₃₀ aryl) ether carboxylic acids, alkylamido ether carboxylic acids; and also the salts of these compounds; and mixtures thereof;
the alkyl and/or acyl groups of these compounds comprise from 6 to 30 carbon atoms, in particular from 12 to 28, better still from 14 to 24, indeed even from 16 to 22, carbon atoms; the aryl group preferably denoting a phenyl or benzyl group;
these compounds possibly being polyoxyalkylenated, notably polyoxyethylenated, and then preferably including from 1 to 50 ethylene oxide units and better still from 2 to 10 ethylene oxide units.

Use may also be made of C₆-C₂₄ alkyl monoesters of polyglycoside-polycarboxylic acids such as C₆-C₂₄ alkyl polyglycoside-citrates, C₆-C₂₄ alkyl polyglycoside-tartrates and C₆-C₂₄ alkyl polyglycoside-sulfosuccinates, and salts thereof.

Preferentially, the carboxylate anionic surfactants are chosen, alone or as a mixture, from:
fatty acids;
acyl glutamates, notably C₆-C₂₄ or even C₁₂-C₂₀ acyl glutamates, such as stearoyl glutamates, and in particular disodium stearoyl glutamate;
acyl sarcosinates, notably C₆-C₂₄ or even C₁₂-C₂₀ acyl sarcosinates, such as palmitoyl sarcosinates, and in particular sodium palmitoyl sarcosinate;
acyl lactylates, notably C₁₂-C₂₈ or even C₁₄-C₂₄ acyl lactylates, such as behenoyl lactylates, and in particular sodium behenoyl lactylate;
C₆-C₂₄ and notably C₁₂-C₂₀ acyl glycinates;
(C₆-C₂₄)alkyl ether carboxylates, and notably (C₁₂-C₂₀)alkyl ether carboxylates;
polyoxyalkylenated (C₆-C₂₄)alkyl(amido) ether carboxylic acids, in particular those including from 2 to 50 ethylene oxide groups;
in particular in the form of alkali metal or alkaline-earth metal, ammonium or amino alcohol salts.

Among the above carboxylic surfactants, mention may be made most particularly of surfactants of fatty acid type, notably of C₆-C₃₀. These surfactants are preferably chosen from the compounds of formula (a) below:

R—C(O)—OX (a)
with
X denoting a hydrogen atom, an ammonium ion, an ion resulting from an alkali metal or alkaline earth metal or an ion resulting from an organic amine, preferably a hydrogen atom, and
R denoting a linear or branched, saturated or unsaturated alkyl group of 5 to 29 carbon atoms.

Preferably, R denotes a linear or branched, saturated or unsaturated alkyl group of 7 to 23 carbon atoms, preferably of 11 to 21 carbon atoms.

Among the fatty acids, mention may be made of lauric acid, palmitic acid, myristic acid, stearic acid, oleic acid and behenic acid.

The fatty acids are advantageously chosen from palmitic acid, myristic acid, stearic acid, and mixtures thereof.

Among the above carboxylic surfactants, mention may be made most particularly of surfactants of sarcosinate type, notably chosen from (C₆-C₃₀)acyl sarcosinates of formula (I) below:

R—C(O)—N(CH₃)—CH₂—C(O)—OX (I)
with
   X denoting a hydrogen atom, an ammonium ion, an ion resulting from an alkali metal or alkaline earth metal or an ion resulting from an organic amine, preferably a hydrogen atom, and
   R denoting a linear or branched alkyl group of 5 to 29 carbon atoms.

Preferably, R denotes a linear or branched alkyl group of 8 to 24 carbon atoms, preferably of 12 to 20 carbon atoms.

Among the (C₆-C₃₀)acyl sarcosinates of formula (I) that may be used in the present composition, mention may be made of palmitoyl sarcosinates, stearoyl sarcosinates, myristoyl sarcosinates, lauroyl sarcosinates and cocoyl sarcosinates, in acid form or in salified form.

The anionic surfactant(s) of sarcosinate type are advantageously chosen from sodium lauroyl sarcosinate, stearoylsarcosine, myristoylsarcosine, and mixtures thereof, preferably from stearoylsarcosine, myristoylsarcosine, and mixtures thereof.

Among the above carboxylic surfactants, mention may also be made of polyoxyalkylenated alkyl(amido) ether carboxylic acids and salts thereof, in particular those including from 2 to 50 alkylene oxide groups, in particular ethylene oxide groups, such as the compounds sold by Kao under the Akypo names.

The polyoxyalkylenated alkyl(amido) ether carboxylic acids capable of being used are preferably chosen from those of formula (II):

R1-(OC₂H₄)n-OCH₂COOA (II)
in which:
   R1 represents a linear or branched C₆-C₂₄ alkyl or alkenyl radical, a (C₈-C₉)alkylphenyl radical, a radical R₂CONH—CH₂—CH₂— with R2 denoting a linear or branched C₉-C₂₁ alkyl or alkenyl radical;
preferably, R1 is a C₈-C₂₀ and preferably C₈-C₁₈ alkyl radical, and aryl preferably denotes phenyl,
   n is an integer or decimal number (mean value) ranging from 2 to 24 and preferably from 2 to 10,
   A denotes H, ammonium, Na, K, Li, Mg or a monoethanolamine or triethanolamine residue.

Use may also be made of mixtures of compounds of formula (II), in particular mixtures of compounds bearing different groups R1.

The polyoxyalkylenated alkyl(amido) ether carboxylic acids that are particularly preferred are those of formula (II) in which:
R1 denotes a C₁₂-C₁₄ alkyl, cocoyl, oleyl, nonylphenyl or octylphenyl radical,
A denotes a hydrogen or sodium atom, and
n ranges from 2 to 20, preferably from 2 to 10.

Even more preferentially, use is made of the compounds of formula (II) in which R1 denotes a C₁₂ alkyl radical, A denotes a hydrogen or sodium atom and n ranges from 2 to 10.

The sulfonate anionic surfactants that may be used include at least one sulfonate function (—SO₃H or —SO₃⁻).

They may be chosen from the following compounds: alkyl sulfonates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfoacetates, N-acyl taurates, acyl isethionates; alkyl sulfolaurates; and also the salts of these compounds;
the alkyl groups of these compounds including from 6 to 30 carbon atoms, notably from 12 to 28, even better still from 14 to 24 or even from 16 to 22 carbon atoms; the aryl group preferably denoting a phenyl or benzyl group;
these compounds possibly being polyoxyalkylenated, notably polyoxyethylenated, and then preferably including from 1 to 50 ethylene oxide units and better still from 2 to 10 ethylene oxide units.

Preferentially, the sulfonate anionic surfactants are chosen, alone or as a mixture, from:
C₆-C₂₄ and notably C₁₂-C₂₀ alkyl sulfosuccinates, notably lauryl sulfosuccinates;
C₆-C₂₄ and notably C₁₂-C₂₀ alkyl ether sulfosuccinates;
C₆-C₂₄ and notably C₁₂-C₂₀ N-acyl taurates;
(C₆-C₂₄)acyl isethionates, preferably (C₁₂-C₁₈)acyl isethionates; in particular in the form of alkali metal or alkaline-earth metal, ammonium or amino alcohol salts.

Preferably, the anionic surfactant(s) of sulfonate type are chosen from N-acyltaurates, and notably N-acyl N-methyltaurates, acylisethionates, and also salts thereof and mixtures thereof.

More preferentially, the anionic surfactant(s) of sulfonate type are chosen from acylisethionates, and also salts thereof and mixtures thereof.

The sulfate anionic surfactants that may be used include at least one sulfate function (—OSO₃H or —OSO₃⁻).

They can be chosen from the following compounds: alkyl sulfates, alkyl ether sulfates, alkylamido ether sulfates, alkylaryl polyether sulfates, monoglyceride sulfates; as well as the salts of these compounds;
the alkyl groups of these compounds including from 6 to 30 carbon atoms, notably from 12 to 28, even better still from 14 to 24 or even from 16 to 22 carbon atoms; the aryl group preferably denoting a phenyl or benzyl group;
these compounds possibly being (poly)oxyalkylenated, notably (poly)oxyethylenated, and then preferably including from 1 to 50 ethylene oxide units and better still from 1 to 10 ethylene oxide units.

Preferentially, the sulfate anionic surfactants are chosen, alone or as a mixture, from:
alkyl sulfates, in particular C₆-C₂₄, indeed even C₁₂-C₂₀, alkyl sulfates;
alkyl ether sulfates, in particular C₆-C₂₄, indeed even C₁₂-C₂₀, alkyl ether sulfates, preferably comprising from 1 to 20 ethylene oxide units;
in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular sodium or magnesium salts are preferably used.

Preferentially, the anionic surfactant(s) are chosen from:
C₆-C₃₀ and notably C₈-C₂₄ fatty acids;
C₆-C₂₄ and notably C₁₂-C₂₀ alkyl sulfates;
C₆-C₂₄ and notably C₁₂-C₂₀ alkyl ether sulfates; preferably comprising from 1 to 20 ethylene oxide units;
C₆-C₂₄, in particular C₁₂-C₂₀, alkyl sulfosuccinates; in particular lauryl sulfosuccinates;
C₆-C₂₄ and notably C₁₂-C₂₀ alkyl ether sulfosuccinates;
C₆-C₂₄ and notably C₁₂-C₂₀ N-acyltaurates;
(C₆-C₂₄)acylisethionates, preferably (C₁₂-C₁₈)acylisethionates;
C₆-C₂₄ and notably C₁₂-C₂₀ acylsarcosinates; notably palmitoylsarcosinates, stearoylsarcosinates and myristoylsarcosinates;
(C₆-C₂₄)alkyl ether carboxylates, preferably (C₁₂-C₂₀)alkyl ether carboxylates;
polyoxyalkylenated (C₆-C₂₄)alkyl(amido) ether carboxylic acids and their salts, in particular those comprising from 2 to 50 alkylene oxide groups, in particular ethylene oxide groups;
C₆-C₂₄ and notably C₁₂-C₂₀ acylglutamates;
C₆-C₂₄ and notably C₁₂-C₂₀ acyl glycinates;
and also their salts, in particular their alkali metal or alkaline earth metal or zinc, ammonium or amino alcohol salts;
and mixtures thereof.

Advantageously, the anionic surfactant(s) are chosen from carboxylate anionic surfactants, sulfonate anionic surfactants and mixtures thereof.

The anionic surfactant(s) are preferably chosen from C₆-C₃₀ fatty acids, acyl(C₆-C₃₀)glycinates, acyl(C₆-C₃₀)lactylates, acyl(C₆-C₃₀)sarcosinates, acyl(C₆-C₃₀)glutamates, alkyl ether carboxylic acids, alkyl(C₆-C₃₀ aryl) ether carboxylic acids, alkylamido ether carboxylic acids; C₆-C₂₄ and notably C₁₂-C₂₀ alkylsulfosuccinates, notably laurylsulfosuccinates; C₆-C₂₄ and notably C₁₂-C₂₀ alkyl ether sulfosuccinates; C₆-C₂₄ and notably C₁₂-C₂₀ N-acyltaurates, (C₆-C₂₄)acylisethionates, preferably (C₁₂-C₁₈)acylisethionates, and also the salts of these compounds; and mixtures thereof.

According to a preferred embodiment, the composition comprises one or more carboxylate anionic surfactants and one or more sulfonate anionic surfactants.

In one particularly preferred embodiment of the invention, the composition comprises one or more carboxylate anionic surfactants chosen from C₆-C₂₄ and notably C₁₂-C₂₀ N-acylglutamates and one or more sulfonate anionic surfactants chosen from (C₆-C₂₄)acylisethionates, preferably (C₁₂-C₁₈)acylisethionates.

When they are present in the solid composition contained in the packaging article according to the invention, the total content of the anionic surfactant(s) is preferably greater than or equal to 15% by weight, more preferentially ranges from 15% to 45% by weight, even more preferentially ranges from 20% to 40% by weight, better still ranges from 25% to 35% by weight relative to the total weight of the composition.

Preferably, the total content of the anionic surfactant(s) chosen from carboxylate surfactants, sulfonate surfactants and mixtures thereof is greater than or equal to 15% by weight, preferentially ranges from 15% to 45% by weight, more preferentially ranges from 20% to 40% by weight, better still ranges from 25% to 35% by weight relative to the total weight of the composition.

Preferably, the total content of the anionic surfactant(s) chosen from carboxylate surfactants ranges from 5% to 30% by weight, more preferentially from 10% to 25% by weight, and better still from 10% to 20% by weight, relative to the total weight of the composition.

Preferably, the total content of the anionic surfactant(s) chosen from sulfonate surfactants ranges from 5% to 30% by weight, preferably from 8% to 25% by weight, better still from 10% to 20% by weight relative to the total weight of the composition.

### Amphoteric or Zwitterionic Surfactants

The solid composition contained in the packaging article according to the present invention can also comprise one or more amphoteric or zwitterionic surfactants.

In particular, the amphoteric or zwitterionic surfactant(s), which are preferably non-silicone, used in the solid composition according to the present invention may notably be derivatives of optionally quaternized secondary or tertiary aliphatic amines, in which derivatives the aliphatic group is a linear or branched chain comprising from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido(C₁-C₆)alkylbetaines and (C₈-C₂₀)alkylamido(C₁-C₆)alkylsulfobetaines, and mixtures thereof.

Among the optionally quaternized derivatives of secondary or tertiary aliphatic amines that may be used, as defined above, mention may also be made of the compounds having the respective structures (III) and (IV) below:

Rₐ—CONHCH₂CH₂—N⁺(R_{b})(R_{c})—CH₂COO⁻,M⁺,X⁻ (III)
in which formula (III):
   Rₐ represents a C₁₀ to C₃₀ alkyl or alkenyl group derived from an acid RₐCOOH preferably present in hydrolyzed copra oil; preferably, Rₐ represents a heptyl, nonyl or undecyl group;
   R_{b} represents a β-hydroxyethyl group;
   R_{c} represents a carboxymethyl group;
   M⁺ represents a cationic counterion derived from an alkali metal or alkaline-earth metal, such as sodium, an ammonium ion or an ion derived from an organic amine; and
   X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylaryl- sulfonates, in particular methyl sulfate and ethyl sulfate; or alternatively M⁺ and X⁻ are absent;

Rₐ′—CONHCH₂CH₂—N(B)(B′) (IV)
in which formula (IV):
   B represents the group —CH₂CH₂OX′;
   B′ represents the group —(CH₂)_{z}Y′, with z=1 or 2;
   X′ represents the group —CH₂COOH, —CH₂—COOZ′, —CH₂CH₂COOH or CH₂CH₂—COOZ′, or a hydrogen atom;
   Y′ represents the group —COOH, —COOZ′ or —CH₂CH(OH)SO₃H or the group CH₂CH(OH)SO₃—Z′;
   Z′ represents a cationic counterion derived from an alkali metal or alkaline-earth metal, such as sodium, an ammonium ion or an ion derived from an organic amine;
   Rₐ′ represents a C₁₀ to C₃₀ alkyl or alkenyl group of an acid Rₐ′—COOH preferably present in hydrolyzed linseed oil or copra oil; preferably, Rₐ′ is an alkyl group, in particular a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by Rhodia under the trade name Miranol® C2M Concentrate.

Use may also be made of compounds of formula (V):

R_{a″}—NHCH(Y″)—(CH₂)ₙCONH(CH₂)_{n′}—N(R_{d})(Rₑ) (V)
in which formula (V):
   Y″ represents the group —COOH, —COOZ″ or —CH₂—CH(OH)SO₃H or the group CH₂CH(OH)SO₃—Z″;
   R_{d} and Rₑ, independently of one another, represent a C₁ to C₄ alkyl or hydroxyalkyl radical;
   Z″ represents a cationic counterion derived from an alkali metal or alkaline-earth metal, such as sodium, an ammonium ion or an ion derived from an organic amine;
   Rₐ″ represents a C₁₀ to C₃₀ alkyl or alkenyl group of an acid Rₐ″—COOH preferably present in hydrolyzed linseed oil or copra oil; and
   n and n′, independently of one another, denote an integer ranging from 1 to 3.

Among the compounds of formula (V), mention may be made of the compound classified in the CTFA dictionary under the name sodium diethylaminopropyl cocoaspartamide and sold by Chimex under the name Chimexane HB.

These compounds may be used alone or as mixtures.

Among the amphoteric or zwitterionic surfactants mentioned above, use is advantageously made of (C₈-C₂₀)alkylbetaines, such as cocoyl betaine, (C₈-C₂₀)alkylamido(C₃-C₈)alkylbetaines, such as cocamidopropylbetaine, (C₈-C₂₀)alkylamphoacetates, (C₈-C₂₀)alkylamphodiacetates and mixtures thereof; and preferably (C₈-C₂₀)alkylbetaines, (C₈-C₂₀)alkylamido(C₃-C₈)alkylbetaines and mixtures thereof.

Preferably, the amphoteric or zwitterionic surfactant(s) are chosen from (C₈-C₂₀)alkylbetaines, (C₈-C₂₀)alkylamido(C₃-C₈)alkylbetaines and mixtures thereof, better still from (C₈-C₂₀)alkylamido(C₃-C₈)alkylbetaines and mixtures thereof.

When they are present in the solid composition contained in the packaging article according to the invention, the total content of the amphoteric or zwitterionic surfactant(s) preferably ranges from 1% to 30% by weight, more preferentially from 2% to 25% by weight, better still from 5% to 20% by weight, and even better still from 8% to 15% by weight, relative to the total weight of the composition.

In a preferred embodiment, the total content of amphoteric or zwitterionic surfactant(s) chosen from (C₈-C₂₀)alkylbetaines, (C₈-C₂₀)alkylamido(C₃-C₈)alkylbetaines and mixtures thereof ranges from 1% to 30% by weight, preferably from 2% to 25% by weight, more preferentially from 5% to 20% by weight, and better still from 8% to 15% by weight, relative to the total weight of the composition.

### Cationic Surfactants

The solid composition contained in the packaging article according to the invention can comprise one or more cationic surfactants.

The cationic surfactant(s) that may be used in the composition according to the invention are generally chosen from optionally polyoxyalkylenated primary, secondary or tertiary fatty amines, quaternary ammonium salts, and mixtures thereof.

The fatty amines generally comprise at least one C8-C30 hydrocarbon chain. Among the fatty amines that may be used according to the invention, examples that may be mentioned include stearylamidopropyldimethylamine and distearylamine.

Examples of quaternary ammonium salts that may notably be mentioned include:
those corresponding to the general formula (X) below:

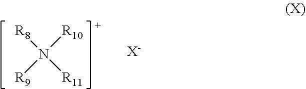

in which the groups R₈ to R₁₁, which may be identical or different, represent a linear or branched aliphatic group including from 1 to 30 carbon atoms, or an aromatic group such as aryl or alkylaryl, at least one of the groups R₈ to R₁₁ including from 8 to 30 carbon atoms and preferably from 12 to 24 carbon atoms. The aliphatic groups may include heteroatoms notably such as oxygen, nitrogen, sulfur and halogens.

The aliphatic groups are chosen, for example, from C1-C30 alkyl, C1-C30 alkoxy, polyoxy(C2-C6)alkylene, C1-C30 alkylamide, (C12-C22)alkylamido(C2-C6)alkyl, (C12-C22)alkyl acetate and C1-C30 hydroxyalkyl groups, X⁻ is an anion chosen from the group of halides, phosphates, acetates, lactates, (C1-C4)alkyl sulfates, (C1-C4)alkylsulfonates or (C1-C4)alkylarylsulfonates.

Among the quaternary ammonium salts of formula (X), preference is given, firstly, to tetraalkylammonium chlorides, for instance dialkyldimethylammonium or alkyltrimethylammonium chlorides in which the alkyl group comprises approximately from 12 to 22 carbon atoms, in particular behenyltrimethylammonium chloride, distearyldimethylammonium chloride, cetyltrimethylammonium chloride or benzyldimethylstearylammonium chloride, or else, secondly, to distearoylethylhydroxyethylmethylammonium methosulfate, dipalmitoylethylhydroxyethylammonium methosulfate or distearoylethylhydroxyethylammonium methosulfate, or else, finally, to palmitylamidopropyltrimethylammonium chloride or stearamidopropyldimethyl(myristyl acetate)ammonium chloride, sold under the name Ceraphyl® 70 by Van Dyk;
quaternary ammonium salts of imidazoline, for instance those of formula (XI) below:

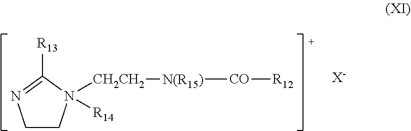

in which R₁₂ represents an alkenyl or alkyl group containing from 8 to 30 carbon atoms, derived, for example, from tallow fatty acids, R₁₃ represents a hydrogen atom, a C₁-C₄ alkyl group or an alkenyl or alkyl group containing from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, (C₁-C₄)alkyl sulfates, (C₁-C₄)alkylsulfonates or (C₁-C₄)alkylarylsulfonates.

Preferably, R₁₂ and R₁₃ denote a mixture of alkenyl or alkyl groups containing from 12 to 21 carbon atoms, for example derived from tallow fatty acids, R₁₄ denotes a methyl group and R₁₅ denotes a hydrogen atom. Such a product is sold, for example, under the name Rewoquat® W 75 by Rewo;
quaternary diammonium or triammonium salts, in particular of formula (XII) below:

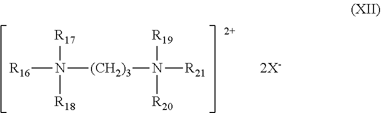

in which R₁₆ denotes an alkyl group containing approximately from 16 to 30 carbon atoms, which is optionally hydroxylated and/or interrupted with one or more oxygen atoms, R₁₇ is chosen from hydrogen, an alkyl group containing from 1 to 4 carbon atoms or a group —(CH₂)₃—N+(R₁₆ₐ)(R₁₇ₐ)(R₁₈ₐ), R₁₆ₐ, R₁₇ₐ, R₁₈ₐ, R₁₈, R₁₉, R₂₀ and R₂₁, which may be identical or different, are chosen from hydrogen or an alkyl group containing from 1 to 4 carbon atoms, and X⁻ is an anion chosen from the group of halides, acetates, phosphates, nitrates, (C₁-C₄)alkyl sulfates, and (C₁-C₄)alkyl- or (C₁-C₄)alkylarylsulfonates, in particular methyl sulfate and ethyl sulfate.

Such compounds are, for example, Finquat CT-P, sold by Finetex (Quaternium 89), and Finquat CT, sold by Finetex (Quaternium 75);
quaternary ammonium salts containing one or more ester functions, for instance those of formula (XIII) below:

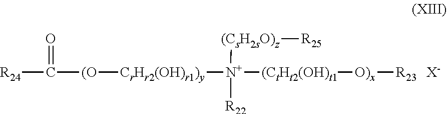

in which: R₂₂ is chosen from C1-C6 alkyl groups and C1-C6 hydroxyalkyl or dihydroxyalkyl groups; R₂₃ is chosen from: the group —C(O)R₂₆, linear or branched, saturated or unsaturated C1-C22 hydrocarbon groups R₂₇, a hydrogen atom; R₂₅ is chosen from: the group —C(O)R₂₈, linear or branched, saturated or unsaturated C1-C6 hydrocarbon groups R₂₉, a hydrogen atom; R₂₄, R₂₆ and R₂₈, which may be identical or different, are chosen from linear or branched, saturated or unsaturated C7-C21 hydrocarbon groups; r, s and t, which may be identical or different, are integers from 2 to 6; r1 and t1, which may be identical or different, are 0 or 1; r2+r1=2 r and t1+t2=2 t, y is an integer from 1 to 10, x and z, which may be identical or different, are integers from 0 to 10, X⁻ is an organic or inorganic, simple or complex anion, with the proviso that the sum x+y+z is from 1 to 15, that when x is 0 R₂₃ denotes R₂₇ and that when z is 0 R₂₅ denotes R₂₉.

The alkyl groups R₂₂ may be linear or branched, and more particularly linear.

Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is a hydrocarbon group R₂₇, it may be long and contain from 12 to 22 carbon atoms, or short and contain from 1 to 3 carbon atoms.

When R₂₅ is a hydrocarbon group R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon groups, and more particularly from linear or branched, saturated or unsaturated C₁₁-C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide, preferably chloride, bromide or iodide, a (C₁-C₄)alkyl sulfate or a (C₁-C₄)alkyl- or (C₁-C₄)alkylarylsulfonate. However, use may be made of methanesulfonate, phosphate, nitrate, tosylate, an anion derived from an organic acid, such as acetate or lactate, or any other anion that is compatible with the ammonium bearing an ester function.

The anion X⁻ is even more particularly chloride, methyl sulfate or ethyl sulfate.

Use is made more particularly, in the composition according to the invention, of the ammonium salts of formula (XIII) in which: R₂₂ denotes a methyl or ethyl group, x and y are equal to 1, z is equal to 0 or 1, r, s and t are equal to 2; R₂₃ is chosen from: the group —C(O)R₂₆, methyl, ethyl or C₁₄-C₂₂ hydrocarbon groups, a hydrogen atom, R₂₅ is chosen from: the group —C(O)R₂₈, a hydrogen atom, R₂₄, R₂₆ and R₂₈, which may be identical or different, are chosen from linear or branched, saturated or unsaturated C₁₃-C₁₇ hydrocarbon groups, and preferably from linear or branched, saturated or unsaturated C₁₃-C₁₇ alkyl and alkenyl groups.

Advantageously, the hydrocarbon groups are linear.

Among the compounds of formula (XIII), examples that may be mentioned include salts, notably the chloride or methyl sulfate, of diacyloxyethyldimethylammonium, diacyloxyethylhydroxyethylmethylammonium, monoacyloxyethyldihydroxyethylmethylammonium, triacyloxyethylmethyl-ammonium or monoacyloxyethylhydroxyethyldimethylammonium, and mixtures thereof. The acyl groups preferably contain 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by quaternization by means of an alkylating agent such as an alkyl halide, preferably a methyl or ethyl halide, a dialkyl sulfate, preferably dimethyl or diethyl sulfate, methyl methanesulfonate, methyl para-toluenesulfonate, glycol chlorohydrin or glycerol chlorohydrin.

Such compounds are sold, for example, under the names Dehyquart® by Henkel, Stepanquat® by Stepan, Noxamium® by CECA or Rewoquat® WE 18 by Rewo-Witco.

The composition according to the invention may contain, for example, a mixture of quaternary ammonium monoester, diester and triester salts with a weight majority of diester salts.

Use may also be made of the ammonium salts containing at least one ester function that are described in patents U.S. Pat. Nos. 4,874,554 and 4,137,180.

Use may also be made of the behenoylhydroxypropyltrimethylammonium chloride sold, for example, by Kao under the name Quartamin BTC 131.

Preferably, the ammonium salts containing at least one ester function contain two ester functions.

Among the cationic surfactants, it is more particularly preferred to choose cetyltrimethylammonium, behenyltrimethylammonium and dipalmitoylethylhydroxyethylmethylammonium salts, and mixtures thereof, and more particularly behenyltrimethylammonium chloride, cetyltrimethylammonium chloride, and dipalmitoylethylhydroxyethylammonium methosulfate, and mixtures thereof.

When they are present in the solid composition contained in the packaging article according to the invention, the total content of the cationic surfactant(s) preferably ranges from 1% to 30% by weight, more preferentially from 2% to 25% weight, and even better still from 3% to 20% by weight, relative to the total weight of the composition.

Preferably, the total content of the cationic surfactant(s) chosen from cetyltrimethylammonium, behenyltrimethylammonium and dipalmitoylethylhydroxyethylmethylammonium salts, and mixtures thereof, preferably ranges from 1% to 30% by weight, more preferentially from 2% to 25% by weight, and even better still from 3% to 20% by weight, relative to the total weight of the composition.

### Non-Ionic Surfactants

The solid composition contained in the packaging article according to the invention can comprise one or more non-ionic surfactants.

The non-ionic surfactant(s) that may be used in the composition of the present invention are notably described, for example, in the “Handbook of Surfactants” by M. R. Porter, published by Blackie & Son (Glasgow and London), 1991, pages 116-178.

Examples of non-ionic surfactants that may be mentioned include the following compounds, alone or as a mixture:
oxyalkylenated (C8-C24)alkylphenols;
saturated or unsaturated, linear or branched, oxyalkylenated or glycerolated C8 to C40 alcohols, preferably including one or two fatty chains;
saturated or unsaturated, linear or branched, oxyalkylenated C8 to C30 fatty acid amides;
esters of saturated or unsaturated, linear or branched, C8 to C30 acids and of polyethylene glycols;
preferably oxyethylenated esters of saturated or unsaturated, linear or branched, C8 to C30 acids and of sorbitol;
fatty acid esters of sucrose;
C8-C30 fatty acid esters of sorbitan, oxyethylenated or non-oxyethylenated;
   (C8-C30)alkyl(poly)glucosides and (C8-C30)alkenyl(poly)glucosides, which are optionally oxyalkylenated (0 to 10 oxyalkylene units) and comprise from 1 to 15 glucose units, (C8-C30)alkyl(poly)glucoside esters;
saturated or unsaturated oxyethylenated plant oils;
condensates of ethylene oxide and/or of propylene oxide;
   N—(C8-C30)alkylglucamine and N—(C8-C30)acylmethylglucamine derivatives;
amine oxides.

They are notably chosen from alcohols, α-diols and (C1-C20)alkylphenols, these compounds being ethoxylated, propoxylated or glycerolated and bearing at least one fatty chain including, for example, from 8 to 24 carbon atoms and preferably from 8 to 18 carbon atoms, the number of ethylene oxide or propylene oxide groups possibly ranging notably from 1 to 200, and the number of glycerol groups possibly ranging notably from 1 to 30.

Mention may also be made of condensates of ethylene oxide and of propylene oxide with fatty alcohols; ethoxylated fatty amides preferably containing from 1 to 30 ethylene oxide units, polyglycerolated fatty amides including on average from 1 to 5, and in particular from 1.5 to 4, glycerol groups, ethoxylated fatty acid esters of sorbitan containing from 1 to 30 ethylene oxide units, fatty acid esters of sucrose, fatty acid esters of polyethylene glycol, (C6-C24 alkyl)polyglycosides, oxyethylenated plant oils, N—(C6-C24 alkyl)glucamine derivatives, amine oxides such as (C10-C14 alkyl)amine oxides or N—(C10-C14 acyl)aminopropylmorpholine oxides.

The C₅-C₃₀ and preferably C₁₂-C₂₂ fatty acid esters (notably monoesters, diesters and triesters) of sorbitan may be chosen from:
sorbitan caprylate; sorbitan cocoate; sorbitan isostearate; sorbitan laurate; sorbitan oleate; sorbitan palmitate; sorbitan stearate; sorbitan diisostearate; sorbitan dioleate; sorbitan distearate; sorbitan sesquicaprylate; sorbitan sesquiisostearate; sorbitan sesquioleate; sorbitan sesquistearate; sorbitan triisostearate; sorbitan trioleate; and sorbitan tristearate.

The polyoxyethylenated C8-C30 (preferably C12-C18) fatty acid esters (notably monoesters, diesters and triesters) of sorbitan notably containing from 2 to 20 mol of ethylene oxide may be chosen from polyoxyethylenated esters of C12-C18 fatty acids, in particular lauric, myristic, cetylic or stearic acid, of sorbitan notably containing from 2 to 30 mol of ethylene oxide, such as:
polyoxyethylenated sorbitan monolaurate (4 OE) (Polysorbate-21),
polyoxyethylenated sorbitan monolaurate (20 OE) (Polysorbate-20),
polyoxyethylenated sorbitan monopalmitate (20 OE) (Polysorbate-40),
polyoxyethylenated sorbitan monostearate (20 OE) (Polysorbate-60),
polyoxyethylenated sorbitan monostearate (4 OE) (Polysorbate-61),
polyoxyethylenated sorbitan monooleate (20 OE) (Polysorbate-80),
polyoxyethylenated sorbitan monooleate (5 OE) (Polysorbate-81),
polyoxyethylenated sorbitan tristearate (20 OE) (Polysorbate-65),
polyoxyethylenated sorbitan trioleate (20 OE) (Polysorbate-85).

The polyoxyethylenated C₅-C₃₀ (preferably C₁₂-C₁₅) fatty acid esters (especially monoesters, diesters, triesters and tetraesters) of sorbitan, containing especially from 2 to 20 mol of ethylene oxide, may be chosen from polyoxyethylenated esters, containing especially from 2 to 20 mol of ethylene oxide, of C₁₂-C₁₈ fatty acids, in particular lauric, myristic, cetylic or stearic acid, of sorbitan, such as:
the ester polyoxyethylenated with 20 OE of sorbitan and of cocoic acid (PEG-20 sorbitan cocoate);
the polyoxyethylenated esters (notably containing from 2 to 20 OE) of sorbitan and of isostearic acid (such as PEG-2 sorbitan isostearate; PEG-5 sorbitan isostearate; PEG-20 sorbitan isostearate such as the product sold under the name Nikkol TI 10 V by Nikkol),
the polyoxyethylenated esters (notably containing from 2 to 20 OE) of sorbitan and of lauric acid (such as PEG-10 sorbitan laurate),
the polyoxyethylenated esters (notably containing from 2 to 20 OE) of sorbitan and of oleic acid containing 10 oxyethylene groups (such as PEG-6 sorbitan oleate; PEG-20 sorbitan oleate),
the polyoxyethylenated esters (notably containing from 3 to 20 OE) of sorbitan and of stearic acid (such as PEG-3 sorbitan stearate; PEG-4 sorbitan stearate; PEG-6 sorbitan stearate).

The non-ionic surfactant(s) are preferably chosen from the ethoxylated C₅-C₂₄ fatty alcohols comprising from 1 to 200 ethylene oxide groups, the ethoxylated C₅-C₃₀ fatty acid esters of sorbitan having from 1 to 30 ethylene oxide units, the (C₆-C₂₄ alkyl)polyglycosides and mixtures thereof.

When they are present in the solid composition contained in the packaging article according to the invention, the total content of the non-ionic surfactant(s) preferably ranges from 1% to 30% by weight, more preferentially from 2% to 25% weight, and even better still from 5% to 20% by weight, relative to the total weight of the composition.

In one preferred embodiment, the total content of the non-ionic surfactant(s) chosen from ethoxylated C₅-C₂₄ fatty alcohols comprising from 1 to 200 ethylene oxide groups, ethoxylated C₅-C₃₀ fatty acid esters of sorbitan having from 1 to 30 ethylene oxide units, (C₆-C₂₄ alkyl)polyglycosides and mixtures thereof preferably ranges from 1% to 30% by weight, more preferentially from 2% to 25% by weight, and even better still from 5% to 20% by weight, relative to the total weight of the composition.

### Fillers

The solid composition contained in the packaging article according to the present invention can optionally also comprise one or more fillers.

For the purposes of the present invention, the term “filler” refers to mineral or organic, polymeric or non-polymeric solid particles.

The fillers according to the invention participate in the solubilization or the break down of the solid composition of the invention, in particular in the presence of water. They may also contribute towards improving the cosmetic performance qualities due to the other compounds present in the composition.

Certain fillers may also have “anticaking” properties.

The mineral fillers may be chosen from silicates, for instance mica or clays, notably kaolin.

The non-polymeric organic fillers may be chosen from monosaccharides, for instance trehalose, sorbitol and mannitol.

The polymeric organic fillers may be chosen from polysaccharides. Mention may be made in particular of cyclodextrins, starches, alginates, gellans, guar gums, celluloses and wood meals. Among the polymeric organic fillers, mention may also be made of crosslinked polyvinylpyrrolidones and polyacrylates (for example Aquakeep).

Advantageously, the filler(s) present in the solid composition of the invention are chosen from polymeric organic fillers and mixtures thereof, preferably from cyclodextrins, starches, alginates, gellans, guar gums, celluloses, wood meals, crosslinked polyvinylpyrrolidones, polyacrylates and mixtures thereof, and more preferentially from starches, guar gums and mixtures thereof.

The total content of the filler(s), when they are present in the solid composition contained in the packaging article of the invention, is preferably greater than or equal to 20% by weight, more preferentially greater than or equal to 30% by weight, and better still greater than or equal to 35% by weight, relative to the total weight of the composition. Preferably, the total content of the filler(s), when they are present in the solid composition of the invention, ranges from 20% to 80% by weight, preferably from 25% to 70% by weight, and more preferentially from 30% to 60% by weight, better still 35%-50%, relative to the total weight of the composition.

Preferably, the solid composition contained in the packaging article according to the invention can comprise one or more fillers.

Preferably, the total content of the filler(s) chosen from starches, present in the solid composition of the invention, is preferably greater than or equal to 20% by weight, more preferentially greater than or equal to 30% by weight, and even better still greater than or equal to 35% by weight, relative to the total weight of the composition.

Advantageously, the total content of the filler(s) chosen from starches, present in the solid composition of the invention, ranges from 20% to 80% by weight, preferably from 30% to 70% by weight, more preferentially from 35% to 60% by weight, even more preferentially from 35% to 50% by weight, relative to the total weight of the composition.

### Cationic Polymers

The solid composition contained in the packaging article according to the present invention can optionally also comprise one or more cationic polymers.

For the purposes of the present invention, the term “cationic polymer” means any polymer comprising cationic groups and/or groups that may be ionized to cationic groups. Preferably, the cationic polymer(s) are hydrophilic or amphiphilic.

The cationic polymers are not silicone-based (they do not comprise any Si—O units).

The cationic polymers may be associative or non-associative.

The preferred cationic polymers are chosen from those that contain units including primary, secondary, tertiary and/or quaternary amine groups that may either form part of the main polymer chain or may be borne by a side substituent directly connected thereto.

Preferably, the cationic polymers according to the invention do not comprise any anionic groups or any groups that can be ionized into anionic groups.

The cationic polymers that may be used preferably have a weight-average molar mass (Mw) of between 500 and 5×10⁶ approximately and preferably between 10³ and 3×10⁶ approximately.

Among the cationic polymers, mention may be made more particularly of:
(1) homopolymers or copolymers derived from acrylic or methacrylic esters or amides and including at least one of the units having the following formulae:

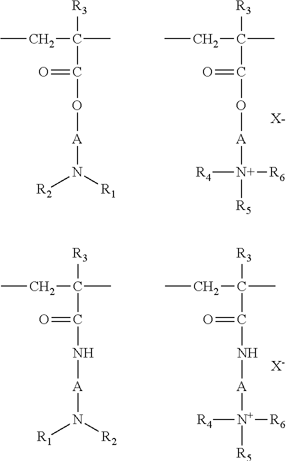

in which formulae:
   R₃, which may be identical or different, denote a hydrogen atom or a CH₃ radical;
   A, which may be identical or different, represent a linear or branched divalent alkyl group of 1 to 6 carbon atoms, preferably 2 or 3 carbon atoms, or a hydroxyalkyl group of 1 to 4 carbon atoms;
   R₄, R₅ and R₆, which may be identical or different, represent an alkyl group containing from 1 to 18 carbon atoms or a benzyl radical, and preferably an alkyl group containing from 1 to 6 carbon atoms;
   R₁ and R₂, which may be identical or different, represent a hydrogen atom or an alkyl group containing from 1 to 6 carbon atoms, preferably methyl or ethyl; and
   X denotes an anion derived from a mineral or organic acid, such as a methosulfate anion or a halide such as chloride or bromide.

The copolymers of family (1) may also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic acids or methacrylic acids or esters thereof, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as the product sold under the name Hercofloc by Hercules,
copolymers of acrylamide and of methacryloyloxyethyltrimethylammonium chloride, such as the products sold under the name Bina Quat P 100 by Ciba Geigy,
   the copolymer of acrylamide and of methacryloyloxyethyltrimethylammonium methosulfate, such as the product sold under the name Reten by Hercules,
quaternized or non-quaternized vinylpyrrolidone/dialkylaminoalkyl acrylate or methacrylate copolymers, such as the products sold under the name Gafquat by ISP, for instance Gafquat 734 or Gafquat 755, or alternatively the products known as Copolymer 845, 958 and 937. These polymers are described in detail in French patents 2077 143 and 2 393 573,
dimethylaminoethyl methacrylate/vinylcaprolactam/vinylpyrrolidone terpolymers, such as the product sold under the name Gaffix VC 713 by ISP,
vinylpyrrolidone/methacrylamidopropyldimethylamine copolymers, such as the products sold under the name Styleze CC 10 by ISP;
quaternized vinylpyrrolidone/dimethylaminopropylmethacrylamide copolymers, such as the product sold under the name Gafquat HS 100 by ISP,
polymers, preferably crosslinked polymers, of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, in particular methylenebisacrylamide. Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by Ciba;
(2) cationic polysaccharides, notably cationic celluloses and galactomannan gums. Among the cationic polysaccharides, mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are notably described in FR 1 492 597, and mention may be made of the polymers sold under the name Ucare Polymer JR (JR 400 LT, JR 125 and JR 30M) or LR (LR 400 and LR 30M) by Amerchol. These polymers are also defined in the CTFA dictionary as quaternary ammoniums of hydroxyethyl cellulose that have reacted with an epoxide substituted with a trimethylammonium group.

Cationic cellulose copolymers or cellulose derivatives grafted with a water-soluble quaternary ammonium monomer are described notably in patent U.S. Pat. No. 4,131,576, and mention may be made of hydroxyalkyl celluloses, for instance hydroxymethyl, hydroxyethyl or hydroxypropyl celluloses notably grafted with a methacryloylethyltrimethylammonium, methacrylamido-propyltrimethylammonium or dimethyldiallylammonium salt. The commercial products corresponding to this definition are more particularly the products sold under the names Celquat L 200 and Celquat H 100 by National Starch.

Among the cationic cellulose derivatives, use may also be made of cationic associative celluloses, which may be chosen from quaternized cellulose derivatives, and in particular quaternized celluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24 or even from 10 to 14 carbon atoms; or mixtures thereof.

Preferentially, mention may be made of the hydroxyethylcelluloses of formula (Ib):

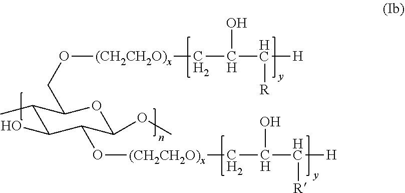

in which:
   R represents an ammonium group RaRbRcN⁺—, Q⁻ in which Ra, Rb and Rc, which may be identical or different, represent a hydrogen atom or a linear or branched C₁ to C₃₀ alkyl, preferably an alkyl, and Q⁻ represents an anionic counterion such as a halide, for instance a chloride or bromide;
   R′ represents an ammonium group R′aR′bR′cN⁺—, Q′⁻ in which R′a, R′b and R′c, which may be identical or different, represent a hydrogen atom or a linear or branched C₁ to C₃₀ alkyl, preferably an alkyl, and Q′⁻ represents an anionic counterion such as a halide, for instance a chloride or bromide;
it being understood that at least one of the radicals Ra, Rb, Rc, R′a, R′b and R′c represents a linear or branched C₈ to C₃₀ alkyl;
   n, x and y, which may be identical or different, represent an integer of between 1 and 10 000.

Preferably, in formula (Ib), at least one of the radicals Ra, Rb, Rc, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radical(s) represent a linear or branched C₁-C₄ alkyl, notably methyl.

Preferably, in formula (Ib), only one of the radicals Ra, Rb, Rc, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radicals represent a linear or branched C₁ to C₄ alkyl, notably methyl.

Even better still, R may be a group chosen from —N⁺(CH₃)₃, Q′⁻ and
—N⁺(C₁₂H₂₅)(CH₃)₂, Q′⁻, preferably a group —N⁺(CH₃)₃, Q′⁻.

Even better still, R′ may be a group —N⁺(C₁₂H₂₅)(CH₃)₂, Q′⁻.

The aryl radicals preferably denote phenyl, benzyl, naphthyl or anthryl groups.

Mention may notably be made of the polymers having the following INCI names:
Polyquaternium-24, such as the product Quatrisoft LM 200®, sold by Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (C₁₈ alkyl), such as the product Crodacel QS®, sold by Croda.

Mention may also be made of the hydroxyethylcelluloses of formula (Ib) in which R represents a trimethylammonium halide and R′ represents a dimethyldodecylammonium halide, preferentially R represents trimethylammonium chloride (CH₃)₃N⁺—, Cl⁻ and R′ represents dimethyldodecylammonium chloride (CH₃)₂(C₁₂H₂₅)N⁺—, Cl⁻. This type of polymer is known under the INCI name Polyquaternium-67; as commercial products, mention may be made of the Softcat Polymer SL® polymers, such as SL-100, SL-60, SL-30 and SL-5, from Amerchol/Dow Chemical.

More particularly, the polymers of formula (Ib) are, for example, those of which the viscosity is between 2000 and 3000 cPs (2 Pa·s and 3 Pa·s) inclusive, preferentially between 2700 and 2800 cPs (2.7 Pa·s and 2.8 Pa·s). Typically, Softcat Polymer SL-5 has a viscosity of 2500 cPs (2.5 Pa·s), Softcat Polymer SL-30 has a viscosity of 2700 cPs (2.7 Pa·s), Softcat Polymer SL-60 has a viscosity of 2700 cPs (2.7 Pa·s) and Softcat Polymer SL-100 has a viscosity of 2800 cPs (2.8 Pa·s). Use may also be made of Softcat Polymer SX-1300X with a viscosity of between 1000 and 2000 cPs (1 Pa·s and 2 Pa·s).

The cationic galactomannan gums are described more particularly in patents U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Such products are notably sold under the names Jaguar C13 S, Jaguar C 15, Jaguar C 17 or Jaguar C162 by Rhodia;
(3) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers;
(4) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyaminoamides can be alkylated or, if they include one or more tertiary amine functions, they can be quaternized;
(5) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical includes from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by Sandoz;
(6) polymers obtained by reacting a polyalkylene polyamine including two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by Hercules Inc. or under the name PD 170 or Delsette 101 by Hercules in the case of the adipic acid/epoxypropyl/diethylenetriamine copolymer;
(7) cyclopolymers of alkyldiallylamine or of dialkyldiallylammonium, such as homopolymers or copolymers including, as main constituent of the chain, units corresponding to formula (VI) or (VII):

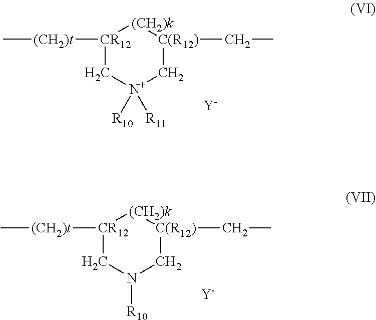

in which formulae (VI) and (VII):
   k and t are equal to 0 or 1, the sum k+t being equal to 1;
   R₁₂ denotes a hydrogen atom or a methyl radical;
   R₁₀ and R₁₁, independently of one another, denote an alkyl group containing from 1 to 6 carbon atoms, a hydroxyalkyl group in which the alkyl group contains 1 to 5 carbon atoms, a C₁ to C₄ amidoalkyl group; or alternatively R₁₀ and R₁₁ may denote, together with the nitrogen atom to which they are attached, heterocyclic groups such as piperidinyl or morpholinyl; R₁₀ and R₁₁, independently of one another, preferably denote an alkyl group containing from 1 to 4 carbon atoms; and
   Y⁻ is an anion such as bromide, chloride, acetate, borate, citrate, tartrate, bisulfate, bisulfite, sulfate or phosphate.

Mention may be made more particularly of the dimethyldiallylammonium salt (for example chloride) homopolymer sold, for example, under the name Merquat 100 by Nalco (and homologues thereof of low weight-average molar masses) and the copolymers of diallyldimethylammonium salts (for example chloride) and of acrylamide, notably sold under the name Merquat 550 or Merquat 7SPR;
(8) quaternary diammonium polymers comprising repeating units of formula:

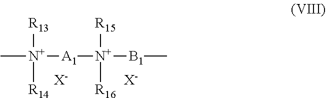

in which formula (VIII):
   R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally comprising a second non-nitrogen heteroatom, or alternatively R₁₃, R₁₄, R₁₅ and R₁₆ represent a linear or branched C₁ to C₆ alkyl radical substituted with a nitrile, ester, acyl or amide group or a group —CO—O—R₁₇-D or —CO—NH—R₁₇-D where R₁₇ is an alkylene and D is a quaternary ammonium group;
   A₁ and B₁ represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups; and
   X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a linear or branched, saturated or unsaturated alkylene or hydroxyalkylene radical, B₁ can also denote a group (CH₂)ₙCO-D-OC—(CH₂)ₙ— in which D denotes:
   a) a glycol residue of formula —O—Z—O—, in which Z denotes a linear or branched hydrocarbon radical or a group corresponding to one of the following formulae: —(CH₂—CH₂—O)ₓ—CH₂—CH₂— and —[CH₂CH(CH₃)—O]_{y}—CH₂—CH(CH₃)—, where x and y denote an integer from 1 to 4, representing a defined and unique degree of polymerization or any number from 1 to 4 representing an average degree of polymerization;
   b) a bis-secondary diamine residue, such as a piperazine derivative;
   c) a bis-primary diamine residue of formula: —NH—Y—NH—, where Y denotes a linear or branched hydrocarbon radical, or alternatively the divalent radical —CH₂—CH₂—S—S—CH₂—CH₂—; or
   d) a ureylene group of formula: —NH—CO—NH—.

Preferably, X⁻ is an anion, such as chloride or bromide. These polymers have a number-average molar mass (Mn) generally of between 1000 and 100 000.

Mention may be made more particularly of polymers which are constituted of repeating units corresponding to the formula:

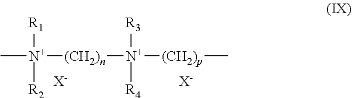

in which formula (IX) R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

A compound of formula (IX) that is particularly preferred is the one for which R₁, R₂, R₃ and R₄ represent a methyl radical and n=3, p=6 and X═Cl, which is known as Hexadimethrine chloride according to the INCI (CTFA) nomenclature;
(9) polyquaternary ammonium polymers comprising units of formula (X):

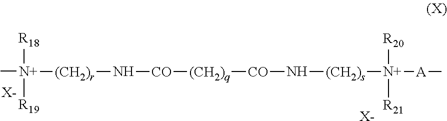

in which formula (X):
   R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom,
   r and s, which may be identical or different, are integers between 1 and 6,
   q is equal to 0 or to an integer between 1 and 34,
   X⁻ denotes an anion, such as a halide, and
   A denotes a dihalide radical or preferably represents —CH₂—CH₂—O—CH₂—CH₂—.

Examples that may be mentioned include the products Mirapol® A 15, Mirapol® AD1, Mirapol® AZ1 and Mirapol® 175 sold by Miranol;
(10) quaternary polymers of vinylpyrrolidone and of vinylimidazole, for instance the products sold under the names Luviquat® FC 905, FC 550 and FC 370 by BASF;
(11) polyamines such as Polyquart® H sold by Cognis, which is referenced under the name Polyethylene Glycol (15) Tallow Polyamine in the CTFA dictionary;
(12) polymers including in their structure:
(a) one or more units corresponding to formula (A) below:

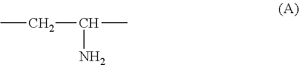

(b) optionally one or more units corresponding to formula (B) below:

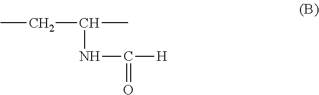

In other words, these polymers may be notably chosen from homopolymers or copolymers including one or more units derived from vinylamine and optionally one or more units derived from vinylformamide.

Preferably, these cationic polymers are chosen from polymers including, in their structure, from 5 mol % to 100 mol % of units corresponding to formula (A) and from 0 to 95 mol % of units corresponding to formula (B), preferentially from 10 mol % to 100 mol % of units corresponding to formula (A) and from 0 to 90 mol % of units corresponding to formula (B).

These polymers may be obtained, for example, by partial hydrolysis of polyvinylformamide. This hydrolysis may take place in acidic or basic medium.

The weight-average molecular mass of said polymer, measured by light scattering, may range from 1000 to 3 000 000 g/mol, preferably from 10 000 to 1 000 000 and more particularly from 100 000 to 500 000 g/mol.

The cationic charge density of these polymers may range from 2 meq/g to 20 meq/g, preferably from 2.5 to 15 meq/g and more particularly from 3.5 to 10 meq/g.

The polymers including units of formula (A) and optionally units of formula (B) are notably sold under the name Lupamin by BASF, for instance, in a non-limiting manner, the products sold under the names Lupamin 9095, Lupamin 5095, Lupamin 1095, Lupamin 9030 (or Luviquat 9030) and Lupamin 9010.

Preferably, the cationic polymer(s) are chosen from cationic polysaccharides (family (2)) and mixtures thereof, more preferentially from cationic galactomannan gums, in particular cationic guar gums.

Preferably, the solid composition according to the invention comprises one or more cationic polymers, preferentially chosen from cationic polysaccharides (family (2)) and mixtures thereof, more preferentially from cationic galactomannan gums, in particular cationic guar gums.

The total content of the cationic polymer(s), when they are present in the solid composition contained in the packaging article according to the invention, is preferably greater than or equal to 0.05% by weight, more preferentially ranges from 0.05% to 5% by weight, and better still from 0.1% to 2% by weight, or even from 0.2% to 1.5% by weight, relative to the total weight of the composition.

Better still, the total content of the cationic polysaccharide(s), when they are present in the solid composition according to the invention, is preferably greater than or equal to 0.05% by weight, more preferentially ranges from 0.05% to 5% by weight, and better still from 0.1% to 2% by weight, or even from 0.2% to 1.5% by weight, relative to the total weight of the composition.

### Silicones

The solid composition contained in the packaging article according to the present invention can optionally comprise one or more silicones.

The silicones that may be used in the invention are different from the cationic polymers defined above.

The silicones that may be used may be soluble or insoluble in the composition according to the invention; they may be in the form of oil, wax, resin or gum; silicone oils and gums are preferred.

Silicones are notably described in detail in Walter Noll's Chemistry and Technology of Silicones (1968), Academic Press.

The volatile silicones may be chosen from those with a boiling point of between 60° C. and 260° C. (at atmospheric pressure) and more particularly from:
i) cyclic polydialkylsiloxanes including from 3 to 7 and preferably 4 to 5 silicon atoms, such as
   octamethylcyclotetrasiloxane and decamethylcyclopentasiloxane.

Mention may be made of the products sold under the name Volatile Silicone 7207 by Union Carbide or Silbione 70045 V 2 by Rhodia, Volatile Silicone 7158 by Union Carbide or Silbione 70045 V 5 by Rhodia;
cyclocopolymers of the dimethylsiloxane/methylalkylsiloxane type having the chemical structure:

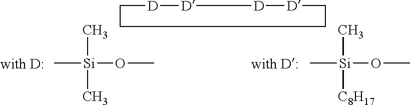

Mention may be made of Volatile Silicone FZ 3109 sold by Union Carbide.
mixtures of cyclic silicones with silicon-derived organic compounds, such as the mixture of octamethylcyclotetrasiloxane and of tetratrimethylsilylpentaerythritol (50/50) and the mixture of octamethylcyclotetrasiloxane and of 1,1′-oxy(2,2,2′,2′,3,3′-hexatrimethylsilyloxy)bisneopentane;
ii) linear polydialkylsiloxanes having 2 to 9 silicon atoms, which generally have a viscosity of less than or equal to 5×10⁻⁶ m²/s at 25° C., such as decamethyltetrasiloxane.

Other silicones belonging to this category are described in the article published in Cosmetics and Toiletries, Vol. 91, January 76, pages 27-32—Todd & Byers Volatile silicone fluids for cosmetics; mention may be made of the product sold under the name SH 200 by Toray Silicone.

Among the non-volatile silicones, mention may be made, alone or as a mixture, of polydialkylsiloxanes and notably polydimethylsiloxanes (PDMS), polydiarylsiloxanes, polyalkylarylsiloxanes, silicone gums and resins, and also organopolysiloxanes (or organomodified polysiloxanes, or alternatively organomodified silicones) which are polysiloxanes including in their structure one or more organofunctional groups, generally attached via a hydrocarbon group, and preferably chosen from aryl groups, amine groups, alkoxy groups and polyoxyethylene or polyoxypropylene groups.

The organomodified silicones may be polydiarylsiloxanes, notably polydiphenylsiloxanes, and polyalkylarylsiloxanes functionalized with the organofunctional groups mentioned previously. The polyalkylarylsiloxanes are particularly chosen from linear and/or branched polydimethyl/methylphenylsiloxanes and polydimethyl/diphenylsiloxanes.

Among the organomodified silicones, mention may be made of organopolysiloxanes including:
polyoxyethylene and/or polyoxypropylene groups optionally including C₆-C₂₄ alkyl groups, such as dimethicone copolyols, and notably those sold by Dow Corning under the name DC 1248 or the oils Silwet® L 722, L 7500, L 77 and L 711 from Union Carbide; or alternatively (C12)alkylmethicone copolyols, and notably those sold by Dow Corning under the name Q2-5200;
substituted or unsubstituted amine groups, in particular C1-C4 aminoalkyl groups; mention may be made of the products sold under the name GP4 Silicone Fluid and GP7100 by Genesee, or under the names Q2-8220 and DC929 or DC939 by Dow Corning;
thiol groups, such as the products sold under the names GP 72 A and GP 71 from Genesee;
alkoxylated groups, such as the product sold under the name Silicone Copolymer F-755 by SWS Silicones and Abil Wax® 2428, 2434 and 2440 by Goldschmidt;
hydroxylated groups, for instance polyorganosiloxanes bearing a hydroxyalkyl function;
acyloxyalkyl groups, such as the polyorganosiloxanes described in patent U.S. Pat. No. 4,957,732;
anionic groups of the carboxylic acid type, as described, for example, in EP 186 507, or of the alkylcarboxylic type, such as the product X-22-3701E from Shin-Etsu; or alternatively of the 2-hydroxyalkylsulfonate or 2-hydroxyalkylthiosulfate type, such as the products sold by Goldschmidt under the names Abil® S201 and Abil® S255;
hydroxyacylamino groups, such as the polyorganosiloxanes described in patent application EP342834; mention may be made, for example, of the product Q2-8413 from Dow Corning.

The silicones may also be chosen from polydialkylsiloxanes, among which mention may be made mainly of polydimethylsiloxanes bearing trimethylsilyl end groups. Among these polydialkylsiloxanes, mention may be made of the following commercial products:
the Silbione® oils of the 47 and 70 047 series or the Mirasil® oils sold by Rhodia, for instance the oil 70 047 V 500 000;
the oils of the Mirasil® series sold by Rhodia;
the oils of the 200 series from Dow Corning, such as DC200, with a viscosity of 60 000 mm²/s;
the Viscasil® oils from General Electric and certain oils of the SF series (SF 96, SF 18) from General Electric.

Mention may also be made of polydimethylsiloxanes bearing dimethylsilanol end groups, known under the name dimethiconol (CTFA), such as the oils of the 48 series from Rhodia.

In this category of polydialkylsiloxanes, mention may also be made of the products sold under the names Abil Wax® 9800 and 9801 by Goldschmidt, which are poly(C1-C20)dialkylsiloxanes.

Products that may be used more particularly in accordance with the invention are mixtures such as:
mixtures formed from a polydimethylsiloxane with a hydroxy-terminated chain, or dimethiconol (CTFA), and from a cyclic polydimethylsiloxane, also known as cyclomethicone (CTFA), such as the product Q2-1401 sold by Dow Corning.

The polyalkylarylsiloxanes are chosen particularly from linear and/or branched polydimethyl/methylphenylsiloxanes and polydimethyl/diphenylsiloxanes with a viscosity ranging from 1×10-5 to 5×10-2 m²/s at 25° C. The polyalkylarylsiloxanes are chosen particularly from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, mention may be made of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 or SF 1265.

The silicones that may be used may be amino silicones. The term “amino silicone” denotes any silicone including at least one primary, secondary or tertiary amine or a quaternary ammonium group.

The weight-average molecular masses of these amino silicones may be measured by gel permeation chromatography (GPC) at ambient temperature (25° C.), as polystyrene equivalent. The columns used are μ styragel columns. The eluent is THF and the flow rate is 1 ml/min. 200 μl of a 0.5% by weight solution of silicone in THF are injected. Detection is performed by refractometry and UV-metry.

Preferably, the amino silicone(s) that may be used in the context of the invention are chosen from:
a) the polysiloxanes corresponding to formula (A):

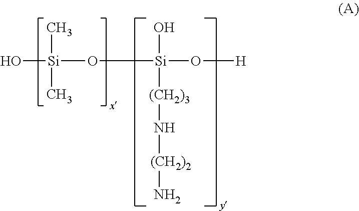

in which x′ and y′ are integers such that the weight-average molecular weight (Mw) is between 5000 and 500 000 approximately;
b) the amino silicones corresponding to formula (B):

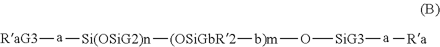

in which:
   G, which may be identical or different, denotes a hydrogen atom or a group from among phenyl, OH, C₁-C₈ alkyl, for example methyl, or C₁-C₈ alkoxy, for example methoxy;
   a, which may be identical or different, denotes 0 or an integer from 1 to 3, in particular 0,
   b denotes 0 or 1, in particular 1,
   m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10;
   R′, which may be identical or different, denotes a monovalent radical of formula —CqH2qL in which q is a number ranging from 2 to 8 and L is an optionally quaternized amine group chosen from the following groups:

—N(R″)₂;—N⁺(R″)₃A⁻;—NR″-Q-N(R″)₂and —NR″-Q-N⁺(R″)₃A⁻,
in which R″, which may be identical or different, denotes hydrogen, phenyl, benzyl, or a saturated monovalent hydrocarbon radical, for example a C₁-C₂₀ alkyl radical; Q denotes a linear or branched group of formula CrH2r, r being an integer ranging from 2 to 6, preferably from 2 to 4; and A⁻ represents a cosmetically acceptable anion, notably a halide such as fluoride, chloride, bromide or iodide.

Preferably, the amino silicones are chosen from the amino silicones of formula (B). Preferably, the amino silicones of formula (B) are chosen from the amino silicones corresponding to formulae (C), (D), (E), (F) and/or (G) below.

Thus, the amino silicones corresponding to formula (B) can be chosen from, alone or as a mixture:

A/ the “trimethylsilyl amodimethicone” silicones corresponding to formula (C):

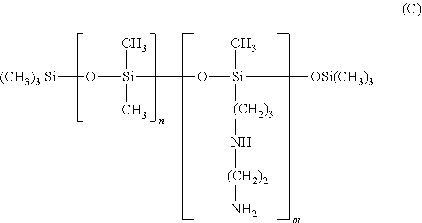

in which m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10.
B/ the silicones of formula (D) below:

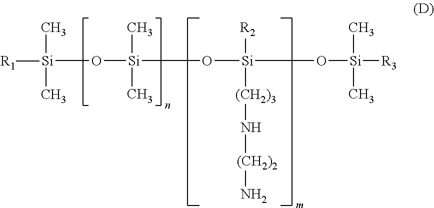

in which:
   m and n are numbers such that the sum (n+m) ranges from 1 to 1000, in particular from 50 to 250 and more particularly from 100 to 200; n possibly denoting a number from 0 to 999, notably from 49 to 249 and more particularly from 125 to 175, and m possibly denoting a number from 1 to 1000, notably from 1 to 10 and more particularly from 1 to 5;
   R₁, R₂ and R₃, which may be identical or different, represent a hydroxyl or C₁-C₄ alkoxy radical, at least one of the radicals R₁ to R₃ denoting an alkoxy radical.

Preferably, the alkoxy radical is a methoxy radical.

The hydroxy/alkoxy mole ratio preferably ranges from 0.2:1 to 0.4:1 and preferably from 0.25:1 to 0.35:1 and more particularly is equal to 0.3:1.

The weight-average molecular mass (Mw) of these silicones preferably ranges from 2000 to 1 000 000 and more particularly from 3500 to 200 000.
C/ the silicones of formula (E) below:

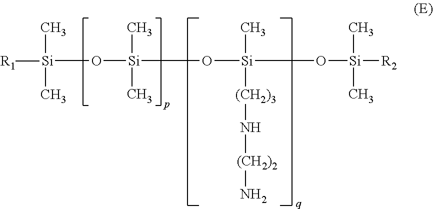

in which:
   p and q are numbers such that the sum (p+q) ranges from 1 to 1000, in particular from 50 to 350 and more particularly from 150 to 250; p possibly denoting a number from 0 to 999, notably from 49 to 349 and more particularly from 159 to 239, and q possibly denoting a number from 1 to 1000, notably from 1 to 10 and more particularly from 1 to 5;
   R₁ and R₂, which are different, represent a hydroxyl or C₁-C₄ alkoxy radical, at least one of the radicals R₁ or R₂ denoting an alkoxy radical.

Preferably, the alkoxy radical is a methoxy radical.

The hydroxy/alkoxy mole ratio generally ranges from 1:0.8 to 1:1.1 and preferably from 1:0.9 to 1:1 and more particularly is equal to 1:0.95.

The weight-average molecular mass (Mw) of the silicone preferably ranges from 2000 to 200 000, even more particularly from 5000 to 100 000 and more particularly from 10 000 to 50 000.

The commercial products comprising silicones of structure (D) or (E) may include in their composition one or more other amino silicones the structure of which is different from formula (D) or (E).

A product containing amino silicones of structure (D) is sold by Wacker under the name Belsil® ADM 652.

A product containing amino silicones of structure (E) is sold by Wacker under the name Fluid WR 1300®.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or non-ionic. The number-average size of the silicone particles in the emulsion generally ranges from 3 nm to 500 nm. Preferably, notably as amino silicones of formula (E), use is made of microemulsions with a mean particle size ranging from 5 nm to 60 nm (limits included) and more particularly from 10 nm to 50 nm (limits included). Thus, use may be made according to the invention of the amino silicone microemulsions of formula (E) sold under the names Finish CT 96 E® or SLM 28020® by Wacker.
D/ the silicones of formula (F) below:

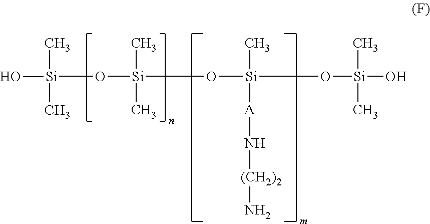

in which:
   m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10;
   A denotes a linear or branched alkylene radical containing from 4 to 8 carbon atoms and preferably 4 carbon atoms. This radical is preferably linear.

The weight-average molecular mass (Mw) of these amino silicones preferably ranges from 2000 to 1 000 000 and even more particularly from 3500 to 200 000.

A silicone corresponding to this formula is, for example, Xiameter MEM 8299 Emulsion from Dow Corning.
E/ the silicones of formula (G) below:

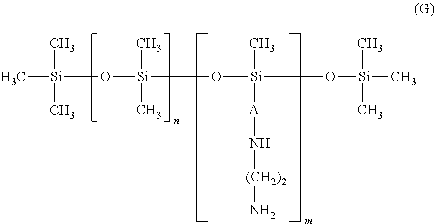

in which:
   m and n are numbers such that the sum (n+m) ranges from 1 to 2000 and in particular from 50 to 150, n possibly denoting a number from 0 to 1,999 and notably from 49 to 149, and m possibly denoting a number from 1 to 2000 and notably from 1 to 10;
   A denotes a linear or branched alkylene radical containing from 4 to 8 carbon atoms and preferably 4 carbon atoms. This radical is preferably branched.

The weight-average molecular mass (Mw) of these amino silicones preferably ranges from 500 to 1 000 000 and even more particularly from 1000 to 200 000.

A silicone corresponding to this formula is, for example, DC2-8566 Amino Fluid from Dow Corning;
c) the amino silicones corresponding to formula (H):

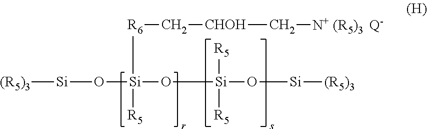

in which:
   R₅ represents a monovalent hydrocarbon radical containing from 1 to 18 carbon atoms, and in particular a C1-C18 alkyl or C2-C18 alkenyl radical, for example methyl;
   R₆ represents a divalent hydrocarbon radical, notably a C1-C18 alkylene radical or a divalent C1-C18, and for example C1-C8, alkylenoxy radical linked to the Si via an SiC bond;
   Q⁻ is an anion such as a halide ion, notably chloride, or an organic acid salt, notably acetate;
   r represents a mean statistical value ranging from 2 to 20 and in particular from 2 to 8;
   s represents a mean statistical value ranging from 20 to 200 and in particular from 20 to 50.

Such amino silicones are notably described in patent U.S. Pat. No. 4,185,087.
d) the quaternary ammonium silicones of formula (I):

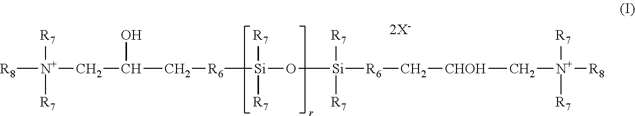

in which:
   R₇, which may be identical or different, represent a monovalent hydrocarbon radical containing from 1 to 18 carbon atoms, and in particular a C₁-C₁₈ alkyl radical, a C₂-C₁₈ alkenyl radical or a ring comprising 5 or 6 carbon atoms, for example methyl;
   R₆ represents a divalent hydrocarbon radical, especially a C₁-C₁₈ alkylene radical or a divalent C₁-C₁₈, and for example C₁-C₈, alkylenoxy radical linked to the Si via an SiC bond;
R₈, which may be identical or different, represent a hydrogen atom, a monovalent hydrocarbon radical containing from 1 to 18 carbon atoms, and in particular a C₁-C₁₈ alkyl radical, a C₂-C₁₈ alkenyl radical or a radical —R6-NHCOR7;
   X⁻ is an anion, such as a halide ion, in particular a chloride ion, or an organic acid salt, in particular an acetate;
   r represents a mean statistical value ranging from 2 to 200 and in particular from 5 to 100.

These silicones are described, for example, in patent EP-A 0 530 974;
e) the amino silicones of formula (J):

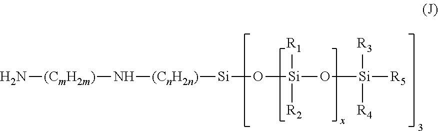

in which:
   R₁, R₂, R₃ and R₄, which may be identical or different, denote a C1-C4 alkyl radical or a phenyl group,
   R₅ denotes a C1-C4 alkyl radical or a hydroxyl group,
   n is an integer ranging from 1 to 5,
   m is an integer ranging from 1 to 5, and
   x is chosen such that the amine number ranges from 0.01 to 1 meq/g;
f) multiblock polyoxyalkylene amino silicones, of the type (AB)n, A being a polysiloxane block and B being a polyoxyalkylene block including at least one amine group.

Said silicones are preferably formed from repeating units having the following general formulae:

[—(SiMe2O)xSiMe2-R—N(R″)—R′—O(C₂H₄O)a(C₃H₆O)b-R′—N(H)—R—]

or alternatively

[—(SiMe2O)xSiMe2-R—N(R″)—R′—O(C₂H₄O)a(C₃H₆O)b-]
in which:
   a is an integer greater than or equal to 1, preferably ranging from 5 to 200 and more particularly ranging from 10 to 100;
   b is an integer between 0 and 200, preferably ranging from 4 to 100 and more particularly between 5 and 30;
   x is an integer ranging from 1 to 10 000 and more particularly from 10 to 5000;
   R″ is a hydrogen atom or a methyl;
      R, which may be identical or different, represent a linear or branched divalent C₂-C₁₂ hydrocarbon radical, optionally including one or more heteroatoms such as oxygen; preferably, R denotes an ethylene radical, a linear or branched propylene radical, a linear or branched butylene radical, or a radical CH₂CH₂CH₂OCH₂CH(OH)CH₂—; preferentially, R denotes a radical CH₂CH₂CH₂OCH₂CH(OH)CH₂—;
      R′, which may be identical or different, represent a linear or branched divalent C₂-C₁₂ hydrocarbon radical, optionally including one or more heteroatoms such as oxygen; preferably, R′ denotes an ethylene radical, a linear or branched propylene radical, a linear or branched butylene radical, or a radical CH₂CH₂CH₂OCH₂CH(OH)CH₂—; preferentially, R′ denotes —CH(CH₃)—CH₂—.

The siloxane blocks preferably represent between 50 mol % and 95 mol % of the total weight of the silicone, more particularly from 70 mol % to 85 mol %.

The amine content is preferably between 0.02 and 0.5 meq/g of copolymer in a 30% solution in dipropylene glycol, more particularly between 0.05 and 0.2.

The weight-average molecular mass (Mw) of the silicone is preferably between 5000 and 1 000 000 and more particularly between 10 000 and 200 000.

Mention may notably be made of the silicones sold under the name Silsoft A-843 or Silsoft A+ by Momentive.
g) and mixtures thereof.

Preferably, the amino silicone(s) are chosen from the amino silicones of formulae (A), (D), (E), (F) and (G), and mixtures thereof.

The silicone(s) are preferably chosen from amino silicones, better still the amino silicones of formulae (A), (D), (E), (F) and (G) above, and mixtures thereof.

When they are present in the solid composition contained in the packaging article according to the invention, the total content of the silicone(s) preferably ranges from 0.1% to 15% by weight, more preferentially from 0.5% to 10% by weight, even more preferentially from 1.5% to 5% by weight relative to the total weight of the composition.

Preferably, the amino silicones may be present in the composition in a total content ranging preferably from 0.1% to 15% by weight, preferably from 0.5% to 10% by weight, more preferentially from 1% to 5% by weight, relative to the total weight of the composition.

### Polyols

The solid composition contained in the packaging article according to the present invention can optionally comprise one or more polyols.

The polyol(s) present in the solid composition of the invention are preferably chosen from the polyols of formula (XIV) below and mixtures thereof:

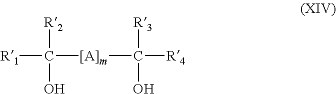

in which formula (XIV):
   R′₁, R′₂, R′₃ and R′₄, which may be identical or different, denote, independently of one another, a hydrogen atom, a linear or branched C₁ to C₆ alkyl radical or a C₁ to C₆ mono- or polyhydroxyalkyl radical,
   A denotes a saturated or unsaturated, linear or branched alkyl radical containing from 1 to 18 carbon atoms, this radical comprising from 0 to 9 oxygen atoms but no hydroxyl group, and
   m denotes 0 or 1.

According to a first embodiment of the invention, the polyol(s) are preferably chosen from the polyols of formula (XIV) in which m has the value 0 and mixtures thereof, and more preferentially from propylene glycol (propane-1,2-diol), 1,2,3-propanetriol, pinacol (2,3-dimethyl-2,3-butanediol), 1,2,3-butanetriol, 2,3-butanediol, sorbitol and mixtures thereof.

According to another embodiment of the invention, the polyol(s) are preferably chosen from the polyols of formula (XIV) in which m has the value 1 and R′₁, R′₂, R′₃ and R′₄, which may be identical or different, denote, independently of one another, a hydrogen atom or a C₁ to C₆ alkyl radical, and mixtures thereof. According to this embodiment, the polyol(s) are advantageously chosen from polyethylene glycols and mixtures thereof, and more particularly the product called PEG-6 or PEG-8 in the CTFA publication (International Cosmetic Ingredient Dictionary, Seventh Edition).

According to yet another embodiment of the invention, the polyol(s) are preferably chosen from the polyols of formula (XIV) in which m has the value 1 and R′₁, R′₂, R′₃ and R′₄, which may be identical or different, denote, independently of one another, a hydrogen atom or a C₁ to C₆ alkyl radical, and the molecular weight of which is less than 200, and mixtures thereof. According to this specific embodiment, the polyol(s) are preferably chosen from 3-methyl-1,3,5-pentanetriol, 1,2,4-butanetriol, 1,5-pentanediol, 2-methyl-1,3-propanediol, 1,3-butanediol, 3-methyl-1,5-pentanediol, neopentyl glycol (2,2-dimethyl-1,3-propanediol), isoprene glycol (3-methyl-1,3-butanediol), hexylene glycol (2-methyl-2,4-pentanediol), and mixtures thereof, and more preferentially from hexylene glycol, neopentyl glycol, 3-methyl-1,5-pentanediol and mixtures thereof.

Preferably, the molecular weight (MW) of said polyol(s) present in the solid composition of the invention is of between 50 and 350, more preferentially between 60 and 200, and even better still between 70 and 100.

Preferably, the polyol(s) are chosen from diols and mixtures thereof, more preferentially from the compounds of formula (XIV) in which R′₁, R′₂, R′₃ and R′₄, which may be identical or different, denote, independently of one another, a hydrogen atom or a C₁ to C₆ alkyl radical, and mixtures thereof.

Advantageously, the polyol(s) are chosen from propylene glycol (propane-1,2-diol), pinacol (2,3-dimethyl-2,3-butanediol), 2,3-butanediol, polyethylene glycols, 1,5-pentanediol, 2-methyl-1,3-propanediol, 1,3-butanediol, 3-methyl-1,5-pentanediol, neopentyl glycol (2,2-dimethyl-1,3-propanediol), isoprene glycol (3-methyl-1,3-butanediol), hexylene glycol (2-methyl-2,4-pentanediol), dipropylene glycol, and mixtures thereof. Preferably, the polyol is dipropylene glycol.

When they are present in the solid composition contained in the packaging article according to the invention, the total content of the polyols(s) preferably ranges from 0.1% to 15% by weight, more preferentially from 0.5% to 10% by weight, and even more preferentially from 1% to 5% by weight, relative to the total weight of the composition.

### Additives

The solid composition according to the present invention may also optionally comprise one or more additives, other than the compounds of the invention and among which mention may be made of anionic, non-ionic or amphoteric polymers or mixtures thereof, antidandruff agents, anti-seborrhoea agents, protein hydrolysates, vitamins and provitamins including panthenol, sunscreens, sequestrants, plasticisers, solubilizers, acidifying agents, alkaline agents, mineral or organic thickeners, notably polymeric thickeners, antioxidants, hydroxy acids and preserving agents.

Of course, those skilled in the art will take care to choose this or these optional additional compounds such that the advantageous properties intrinsically attached to the composition according to the invention are not, or not substantially, detrimentally affected by the envisaged addition(s).

The above additives can generally be present in an amount, for each of them, of between 0% and 20% by weight, relative to the total weight of the composition.

The solid composition contained in the packaging article according to the invention may be a cleansing and/or conditioning composition, notably a hair cleansing and/or conditioning composition.

In a first embodiment, the solid composition contained in the packaging article according to the invention comprises:
one or more anionic surfactants, as defined above,
one or more amphoteric surfactants, as defined above,
optionally one or more non-ionic surfactants, as defined above.

In another embodiment, the solid composition contained in the packaging article according to the invention comprises:
one or more cationic surfactants, as defined above,
optionally one or more amphoteric surfactants and/or one or more non-ionic surfactants, as defined above.

A subject of the present invention is also a process for cosmetic treatment of keratin materials, notably keratin fibres, in particular human keratin fibres such as the hair, comprising a step of using a packaging article as defined hereinabove.

This cosmetic treatment process can in particular be a process for washing and/or conditioning keratin materials, notably keratin fibres, in particular the hair.

Preferably, said cosmetic treatment process comprises the following steps:
i) mixing the packaging article in a composition that is capable of totally or partially solubilizing the envelope of said packaging article,
ii) applying the composition obtained in step i) to the keratin materials, notably keratin fibres,
iii) optionally leaving on said composition,
iv) rinsing said keratin materials, notably keratin fibres, and
v) optionally drying said keratin materials, notably keratin fibres.

Preferably, the composition capable of solubilizing the envelope is water or an aqueous composition.

Thus, the aqueous composition may simply be water. The aqueous composition may optionally comprise at least one polar solvent. Among the polar solvents that may be used in this composition, mention may be made of organic compounds that are liquid at ambient temperature (25° C.) and at least partially water-miscible.

Examples that may be mentioned more particularly include alkanols such as ethyl alcohol and isopropyl alcohol, aromatic alcohols such as benzyl alcohol and phenylethyl alcohol, or polyols or polyol ethers, for instance ethylene glycol monomethyl ether, monoethyl ether and monobutyl ether, propylene glycol or ethers thereof, for instance propylene glycol monomethyl ether, butylene glycol, dipropylene glycol, and also diethylene glycol alkyl ethers, for instance diethylene glycol monoethyl ether or monobutyl ether.

More particularly, if one or more solvents are present, their respective content in the aqueous composition ranges from 0.5% to 20% by weight and preferably from 2% to 10% by weight relative to the weight of said aqueous composition.

The dilution ratio (expressed by weight) between one or more packaging articles, as defined above, and the composition that is suitable for solubilizing the packaging article(s) is preferably between 10/90 and 90/10, and more preferentially between 10/90 and 50/50. Better still, this dilution ratio is 20/80.

In particular, the composition obtained on conclusion of the mixing (step i) of the process) may be applied to wet or dry keratin fibres. It is advantageously left in place on the keratin fibres for a time ranging from 1 to 15 minutes, preferably from 2 to 10 minutes.

The keratin fibres are then rinsed with water. They may optionally be washed with a shampoo, followed by rinsing with water, before being dried or left to dry.

Finally, a subject of the present invention is the use of a packaging article as defined hereinabove for washing and/or conditioning keratin materials, notably keratin fibres, in particular human keratin fibres such as the hair.

The examples that follow serve to illustrate the invention without, however, being limiting in nature.

### EXAMPLES

### Packaging Article Envelope

An envelope composed of two layers A and B forming a unit film according to the present invention is prepared. The layer A comprises wood pulp and carboxymethylcellulose. The layer B comprises polyvinyl alcohol (PVOH) polymers

The characteristics of the layers and of the film are shown in the table below:

TABLE 1

| | | | | Force | Breakup |
|---|---|---|---|---|---|
| | Thick- | Gram- | Water | at | time at |
| | ness | mage | uptake | break | 10° C.* |
| Layer A (wood pulp and | 65 μm | 30 g/m² | 2.9% | 61N | — |
| carboxymethylcellulose) | | | | | |
| Layer B (PVOH) | 15 μm | 20 g/m² | 4% | 13N | — |
| Film (A + B) | 80 μm | — | — | — | Breakup: |
| | | | | | 4 |
| | | | | | seconds |

*determined according to the procedure described in ″Standard Test Method for Solubility of MonoSol ® Water Soluble Film when contained within a Plastic Holder″ (MSTM-205)

### Shampoo Composition

The composition C according to the invention was prepared from the ingredients of which the contents, unless otherwise indicated, are shown as mass percentages of active material relative to the total weight of the composition, in the table below:

TABLE 2

| | Composition C |
|---|---|
| Sodium cocoyl isethionate | 14.3 |
| Sodium lauroyl glutamate | 12.7 |
| Cocamidopropyl betaine | 13.6 |
| Hydroxypropyl guar | 0.8 |
| hydroxypropyltrimonium chloride | |
| Magnesium carbonate | 7 |
| Sodium bicarbonate | 3.75 |
| Citric acid | 1.25 |
| Sodium chloride | 2.4 |
| Fragrance | 3.5 |
| Dipropylene glycol | 1.25 |
| Corn starch | qs 100 |

The composition C in powder form is packaged in the envelope in a proportion of 94 g of composition in a envelope of 6 g.

The packaging article thus obtained solubilizes rapidly (in less than 30 seconds) in the presence of water, notably of water at 37° C.

Once applied to the head of hair, a good start of foaming and an abundant foam are obtained.

Rinsing is then easy, without leaving residues on the head of hair.

1. Packaging article comprising:
an envelope defining at least one cavity, the envelope being composed of at least two layers assembled together in order to form just one unit film,
the first layer comprising at least 60% by weight, relative to the total weight of the first layer, of one or more compounds chosen from cellulose-based polymers, plant fibres and mixtures thereof, and
the second layer comprising nearly 75% by weight, relative to the total weight of the second layer, of one or more vinyl alcohol polymers,
a solid composition comprising less than 10% by weight of water, relative to the total weight of the composition, and one or more surfactants,
it being understood that said solid composition is in at least one of the cavities defined by the envelope.

2. Packaging article according to claim 1, characterized in that the compound(s) chosen from cellulose-based polymers, plant fibres and mixtures thereof are chosen from wood pulp, carboxymethylcelluloses, and also the sodium salts thereof, and mixtures thereof.

3. Packaging article according to claim 1, characterized in that the compound(s) chosen from cellulose-based polymers, plant fibres and mixtures thereof are present in the first layer in a content of greater than or equal to 70% by weight, preferably greater than or equal to 75% by weight, more preferentially greater than or equal to 80% by weight, even more preferentially greater than or equal to 90% by weight and better still greater than or equal to 95% by weight, relative to the total weight of the first layer.

4. Packaging article according to claim 1, characterized in that the vinyl alcohol polymer(s) result from the total or partial hydrolysis of vinyl acetate polymers.

5. Packaging article according to claim 1, characterized in that the degree of hydrolysis of the vinyl acetate polymers is greater than or equal to 75%, preferably greater than or equal to 80%.

6. Packaging article according to claim 1, characterized in that the vinyl alcohol polymer(s) are present in the second layer in a content of greater than or equal to 80% by weight, preferably greater than or equal to 85% by weight, more preferentially greater than or equal to 90% by weight, even more preferentially greater than or equal to 95% by weight, relative to the total weight of the second layer.

7. Packaging article according to claim 1, characterized in that the first layer and/or the second layer additionally comprise one or more plasticisers, notably chosen from polyols.

8. Packaging article according to claim 1, characterized in that the first layer and/or the second layer additionally comprise one or more fillers.

9. Packaging article according to claim 1, characterized in that the ratio between the weight of the first layer and the weight of the second layer ranges from 25/75 to 75/25, preferably from 30/70 to 70/30, more preferentially from 35/65 to 65/35, even more preferentially from 65/35 to 55/45.

10. Packaging article according to claim 1, characterized in that the second layer is adhesive.

11. Packaging article according to claim 1, characterized in that the surfactant(s) are chosen from anionic surfactants, amphoteric or zwitterionic surfactants, cationic surfactants, non-ionic surfactants, and/or mixtures thereof.

12. Packaging article according to claim 1, characterized in that the solid composition also comprises one or more fillers, preferably chosen from polymeric organic fillers and mixtures thereof, more preferentially from cyclodextrins, starches, alginates, gellans, guar gums, celluloses, wood meals, crosslinked polyvinylpyrrolidones, polyacrylates and mixtures thereof, and even more preferentially from starches, guar gums and mixtures thereof.

13. Packaging article according to claim 1, characterized in that the solid composition also comprises one or more polyols, preferably chosen from propylene glycol (propane-1,2-diol), pinacol (2,3-dimethyl-2,3-butanediol), 2,3-butanediol, polyethylene glycols, 1,5-pentanediol, 2-methyl-1,3-propanediol, 1,3-butanediol, 3-methyl-1,5-pentanediol, neopentyl glycol (2,2-dimethyl-1,3-propanediol), isoprene glycol (3-methyl-1,3-butanediol), hexylene glycol (2-methyl-2,4-pentanediol), dipropylene glycol, and mixtures thereof; more preferentially, the polyol is dipropylene glycol.

14. Packaging article according to claim 1, characterized in that the solid composition comprises:
one or more anionic surfactants,
one or more amphoteric surfactants,
optionally one or more non-ionic surfactants.

15. Packaging article according to claim 1, characterized in that the solid composition comprises:
one or more cationic surfactants,
optionally one or more amphoteric surfactants and/or one or more non-ionic surfactants.

16. Process for cosmetic treatment of keratin fibres, in particular human keratin fibres such as the hair, comprising a step of using a packaging article according to claim 1; preferably, said cosmetic treatment process comprises the following steps:
i) mixing the packaging article in a composition that is capable of dissolving, totally or partially, the envelope of said packaging article,
ii) applying the composition obtained in step i) to the keratin materials, notably keratin fibres,
iii) optionally leaving on said composition,
iv) rinsing said keratin materials, notably keratin fibres, and
v) optionally drying said keratin materials, notably keratin fibres.

17. Use of a packaging article according to claim 1, for washing and/or conditioning keratin materials, notably keratin fibres, in particular human keratin fibres such as the hair.
